SEQUENCE LISTING (112 sequences)

SEQ ID NO: 1 - DNA, 3765 bp, E. coli
  CDS (1)..(3765)
  NZ_AAJV01000028
atggatacccgaaatttctggatccgtgattatcttgatttggcacagaataaaggtgtatttcagcctggagcatacggggtaaaaacgccattaaaaaatggtggtgaatttagttttcctgaagtaacgatccctgatttttctccagtatctgctaaaggtgcaacaactgctattggtaatgcctacagtgttaccgcaagccataatggcactattcaccatgccgttaaaacccagacatggggacagtcagactatcattatgttgatcgggtgaccaaaggtgactttgcggtccagcgtctggataagtttgttgttgaaacagcgggtgcaacagagcatgctgatttcaacttatcagcagcagaagcactagagcgttatggtattgagttcaatggaaagaaacagataatcggttttcgggtcggatccggagcaaccggtgttacatcttatggggtgggacagacatataatccattattacgcagtgcttctatgtttcagttaaactggaacaatatgttagccacgaataatacaggtggattttataatgaagtgacaggaggagacagcggttccggattttatctttatgacaatcagagaaaaaaatgggtcattcttggaacaacatatggtaaagcattctccagtaaggatacctgggccttttttgcccgatatgatcagaacactgtcgataccctgaaaaatacttttactcaggaggtgaacctcaatggtcagaaaatgaccgtcaataataaaaatattactgttaacggtaatacaaccgctattgaactgaccaggaataataaaaataaagatttgaaatttcatggtggcgggagcattgaacttaccgataatctgaactcaggaaccggaggactgatttttgatgagggacaacattattcggttattgggaaagataaaacctataaaggggcgggtgttgagatcggaaaaggtacggttgttgactggtcggtaaaaggagcggcaaacgataacctgcacaaaacaggggccgggacactgaatgtcaatgtggcccaggggaataacctgaaaacaggtgacggtaccgtttttcttaatgcagaaaaggctttcaatgctatctatgttgccagtggccgtggaacggtcaaactggggcaggccgatgcgctggataaaaatagtgattacagaggtatttattttaccagccgtggagggactctggatttaaacgggttcagccagtcgtttaagaagattgcggcaactgatgttggtaccattatcaccaatacttctgataaaacagcgaccctttctctacaaaacccttcccgttatgtctatcacggtagtatcacgggaaatacgaatatcgaacacactggaacacagaaaagtgctgacagtagcctgattattgacggaaacattaatacgcacaatgacattactgtacggaattcccagcttcggttacagggacatgccacaacacatgccatttttcgtgatggtccccggcactgctatgtaccgggagtcctttgtgacaaagattttgtcgctgatttcgccagactggagagtgaggcaaacaaaaaaaataacagtgcctataaaacaaataaccaggtggcttcctttgaccagcccgactgggaaacccgacattttcgatttaagacactgaatctggaaaacgcagaattcacaactgcacgtaactcagttgttgagggtgatattgtcgcatcgaattcaacgctgaaactggggggcgacgttccggtattcattgatatgtatgatggcatcaatattaccggtaatggttttggcttccgccaggacgttcgtgaaggacgctcagcagatgatggcagtagcagctatacgggcaatattacactgcagaaaggctccacgctggacattaacaaccgcttcaccggcgggattgaggctcatgacagccaggtaaacgtcacctcaccggatgcccttctgcaaaacagtggtgtcttcatgaactccaccctttctgtccgtgacggcggtcatctgacggcacaaaaagggctctacagtgacggccgggttcagattggaaagaacggtacgctttccctgagcggcacgccggagaatggcgcggataatacctggatgcctgttctgacatacatgacggaaggctacgatttaaccggtgataacgccacgctggacatcagccagcaggcgcatgtttccggggatgttcatgcaaccagttcatccacaattcgtattggctctgaaaacccgggctcagtttcctcttctgtctcccctgttctggctgccggggtgttcagcgggtataacgcggcgtactacggtgccatcaccggcggtaagggaaacgtcagtatgaataatggcctgtggcagctgaccggagattccgatatcaacagtctgacgacccgtaacagccgggttcagtctgaagaaaagggtgccttccgtaccctgacggttaatacacttgatgccacgggcagtgattttgtcctgcgcactgacctgaagggcgctgataaaatcagtattacggagaaagccagcggttcagacaacaccctgaatgtcagctttatgaagaacccgtctccgggacagtccctgaatatcccgctggtcagtgcaccggccggaacatcaggggatatctttaaggccggcacccgggtgactggtttcagtcgtgtgacgccgacgctgcatgtcgacaccacgggtggcagtacgaagtggattctggatggtttcaggacggaagccgataaagcggcagctgcgaaggcggacagtttcatgaatgctggctacaaaaactttatgacggaagttaacaacctgaacaaacgtatgggtgaactgcgtgatacgaacggtgatgccggtgcctgggcccgtatcatgaacggcgcaggttcagccgatggcgggtacagtgataattacactcacgttcaggtcggctttgacaaaaaacatgtgctggatggcgtggacctgtttaccgggatcacaatgacctataccgacagcagtgcagacagtgatgcgttcagcgggaaaacaaaatccgtggggggcggtctgtatgcttcagcattgtttaactccggtgcctacattgatttgattggtaaatacattcaccataacaatgattacacaggcaactttgccggtctgggtacgaagcactacggaacccactcctggtatgccggagcggaaacgggttaccgttatcacctgacggaagacacatttattgagcctcaggccgaactggtttacggcgcagtgtccgggaaaacattccgctggaaagacggtgagatggacctgagtatgaagaacaaggatttcagcccgttgattggcagaacagggattgaactgggcaaaacattcagtggtaaggactggagtgtgacagcccgtgccggaaccagctggcagtttgacctgctgaataatggcgagacggttcttcgtgatgcctccggagaaaaacggattaaaggggagaaggacagcaggatgttgttcaatgtcggcatgaacgcacagataaaggacaacatgcgctttgggctggagtttgagaaatccgcctttggtaaatacaacgtggataacgcgataaacgcgaatttccggtatatgttctga

SEQ ID NO: 2 - protein, 1254 aa, E. coli
  NZ_AAJV01000028
MDTRNFWIRDYLDLAQNKGVFQPGAYGVKTPLKNGGEFSFPEVTIPDFSPVSAKGATTAIGNAYSVTASHNGTIHHAVKTQTWGQSDYHYVDRVTKGDFAVQRLDKFVVETAGATEHADFNLSAAEALERYGIEFNGKKQIIGFRVGSGATGVTSYGVGQTYNPLLRSASMFQLNWNNMLATNNTGGFYNEVTGGDSGSGFYLYDNQRKKWVILGTTYGKAFSSKDTWAFFARYDQNTVDTLKNTFTQEVNLNGQKMTVNNKNITVNGNTTAIELTRNNKNKDLKFHGGGSIELTDNLNSGTGGLIFDEGQHYSVIGKDKTYKGAGVEIGKGTVVDWSVKGAANDNLHKTGAGTLNVNVAQGNNLKTGDGTVFLNAEKAFNAIYVASGRGTVKLGQADALDKNSDYRGIYFTSRGGTLDLNGFSQSFKKIAATDVGTIITNTSDKTATLSLQNPSRYVYHGSITGNTNIEHTGTQKSADSSLIIDGNINTHNDITVRNSQLRLQGHATTHAIFRDGPRHCYVPGVLCDKDFVADFARLESEANKKNNSAYKTNNQVASFDQPDWETRHFRFKTLNLENAEFTTARNSVVEGDIVASNSTLKLGGDVPVFIDMYDGINITGNGFGFRQDVREGRSADDGSSSYTGNITLQKGSTLDINNRFTGGIEAHDSQVNVTSPDALLQNSGVFMNSTLSVRDGGHLTAQKGLYSDGRVQIGKNGTLSLSGTPENGADNTWMPVLTYMTEGYDLTGDNATLDISQQAHVSGDVHATSSSTIRIGSENPGSVSSSVSPVLAAGVFSGYNAAYYGAITGGKGNVSMNNGLWQLTGDSDINSLTTRNSRVQSEEKGAFRTLTVNTLDATGSDFVLRTDLKGADKISITEKASGSDNTLNVSFMKNPSPGQSLNIPLVSAPAGTSGDIFKAGTRVTGFSRVTPTLHVDTTGGSTKWILDGFRTEADKAAAAKADSFMNAGYKNFMTEVNNLNKRMGELRDTNGDAGAWARIMNGAGSADGGYSDNYTHVQVGFDKKHVLDGVDLFTGITMTYTDSSADSDAFSGKTKSVGGGLYASALFNSGAYIDLIGKYIHHNNDYTGNFAGLGTKHYGTHSWYAGAETGYRYHLTEDTFIEPQAELVYGAVSGKTFRWKDGEMDLSMKNKDFSPLIGRTGIELGKTFSGKDWSVTARAGTSWQFDLLNNGETVLRDASGEKRIKGEKDSRMLFNVGMNAQIKDNMRFGLEFEKSAFGKYNVDNAINANFRYMF

SEQ ID NO: 3 - DNA, 29 bp, artificial
  primer SPATE1
gaggtcaacaacctgaacaaacgtatggg

SEQ ID NO: 4 - DNA, 24 bp, artificial
  primer SPATE2
ccggcacgggctgtcactttccag

SEQ ID NO: 5 - DNA, 22 bp, artificial
  primer ChuAf
cggacgaaccaacggtcaggat

SEQ ID NO: 6 - DNA, 22 bp, artificial
  primer ChuAr
tgccgccagtaccaaagacacg

SEQ ID NO: 7 - DNA, 23 bp, artificial
  primer Yjaf
cgtgaagtgtcaggagacgctgc

SEQ ID NO: 8 - DNA, 25 bp, artificial
  primer Yjar
tgcgttcctcaacctgtgacaaacc

SEQ ID NO: 9 - DNA, 23 bp, artificial
  primer Tsp1
gggagtaatgtcggggcattcag

SEQ ID NO: 10 - DNA, 26 bp, artificial
  primer Tsp2
catcgcgccaacaaagtattacgcag

SEQ ID NO: 11 - DNA, 24 bp, artificial
  primer Cnff
agtactgacactcactcaagccgc

SEQ ID NO: 12 - DNA, 29 bp, artificial
  primer Cnfr
gcagaacgacgttcttcataagtatcacc

SEQ ID NO: 13 - DNA, 22 bp, artificial
  primer IpgDf
cgacttctcttctgacgccgac

SEQ ID NO: 14 - DNA, 23 bp, artificial
  primer IpgDr
caacattcctccagcctaagccc

SEQ ID NO: 15 - DNA, 24 bp, artificial
  primer Vt1f
cgcatagtggaacctcactgacgc

SEQ ID NO: 16 - DNA, 21 bp, artificial
  primer Vt1r
catccccgtacgactgatccc

SEQ ID NO: 17 - DNA, 26 bp, artificial
  primer Vt2f
cggaatgcaaatcagtcgtcactcac

SEQ ID NO: 18 - DNA, 21 bp, artificial
  primer Vt2r
tccccgatactccggaagcac

SEQ ID NO: 19 - DNA, 22 bp, artificial
  primer HlyAf
tgcagcctccagtgcatccctc

SEQ ID NO: 20 - DNA, 24 bp, artificial
  primer Hlyar
cttaccactctgactgcgatcagc

SEQ ID NO: 21 - DNA, 21 bp, artificial
  primer Staf
gtgaaacaacatgacgggagg

SEQ ID NO: 22 - DNA, 21 bp, artificial
  primer Star
ataacatccagcacaggcagg

SEQ ID NO: 23 - DNA, 25 bp, artificial
  primer Stbf
ggggttagagatggtactgctggag

SEQ ID NO: 24 - DNA, 25 bp, artificial
  primer Stbr
gacaatgtccgtcttgcgttaggac

SEQ ID NO: 25 - DNA, 22 bp, artificial
  primer LTf
ccgtgctgactctagaccccca

SEQ ID NO: 26 - DNA, 26 bp, artificial
  primer LTr
cctgctaatctgtaaccatcctctgc

SEQ ID NO: 27 - DNA, 24 bp, artificial
  primer Eaef
ccaggcttcgtcacagttgcaggc

SEQ ID NO: 28 - DNA, 23 bp, artificial
  primer Eaer
cgccagtattcgccaccaatacc

SEQ ID NO: 29 - DNA, 25 bp, artificial
  primer Bf1F
gttagtgcccagatgcaggatgcgg

SEQ ID NO: 30 - DNA, 28 bp, artificial
  primer Bf2F
gaactcggtttatgcagttcatggactg

SEQ ID NO: 31 - DNA, 25 bp, artificial
  primer Bf3R
tgggttgtagacatcccactggctt

SEQ ID NO: 32 - DNA, 29 bp, artificial
  primer Bf4R
ggatacatcagctgggttgtagacatccc

SEQ ID NO: 33 - DNA, 21 bp, artificial
  primer PapF
ccggcgttcaggctgtagctg

SEQ ID NO: 34 - DNA, 31 bp, artificial
  primer PapR
gctacagtggcagtatgagtaatgaccgtta

SEQ ID NO: 35 - DNA, 26 bp, artificial
  primer PAI1
tagctcagacgccaggattttccctg

SEQ ID NO: 36 - DNA, 28 bp, artificial
  primer PAI2
cctggcgcctgcgggctgactatcaggg

SEQ ID NO: 37 - DNA, 24 bp, artificial
  primer BmaEf
ctaacttgccatgctgtgacagta

SEQ ID NO: 38 - DNA, 24 bp, artificial
  primer BmaEr
ttatcccctgcgtagttgtgaatc

SEQ ID NO: 39 - DNA, 25 bp, artificial
  primer Sfaf
cggaggagtaattacaaacctggca

SEQ ID NO: 40 - DNA, 25 bp, artificial
  primer Sfar
ctccggagaactgggtgcatcttac

SEQ ID NO: 41 - DNA, 31 bp, artificial
  primer Afaf
tatggtgagttggcggggatgtacagttaca

SEQ ID NO: 42 - DNA, 25 bp, artificial
  primer Afar
ccgggaaagttgtcggatccagtgt

SEQ ID NO: 43 - DNA, 24 bp, artificial
  primer AIDA1
tatgccacctggtatgccgatgac

SEQ ID NO: 44 - DNA, 20 bp, artificial
  primer AIDA2
acgcccacattcccccagac

SEQ ID NO: 45 - DNA, 27 bp, artificial
  primer AggRf
gagttaggtcactctaacgcagagttg

SEQ ID NO: 46 - DNA, 29 bp, artificial
  primer AggRr
gaccaattcggacaactgcaagcatctac

SEQ ID NO: 47 - DNA, 25 bp, artificial
  primer Ag43F
tgacacaggcaatggactatgaccg

SEQ ID NO: 48 - DNA, 20 bp, artificial
  primer Ag43R
ggcatcatcccggaccgtgc

SEQ ID NO: 49 - DNA, 22 bp, artificial
  primer Primer1
caagtcattaatacmaacagcc

SEQ ID NO: 50 - DNA, 20 bp, artificial
  primer Primer2
gacatrttrgavacttcsgt

SEQ ID NO: 51 - DNA, 26 bp, artificial
  primer FimHf
ctggtcattcgcctgtaaaaccgcca

SEQ ID NO: 52 - DNA, 30 bp, artificial
  primer HimHr
gtcacgccaataatcgattgcacattccct

SEQ ID NO: 53 - DNA, 20 bp, artificial
  primer ITSF
gtcgtaacaaggtagccgta

SEQ ID NO: 54 - DNA, 15 bp, artificial
  primer ITSReub
gccaaggcatccacc

SEQ ID NO: 55 - DNA, 20 bp, artificial
  primer 27f
agagtttgatcmtggctcag

SEQ ID NO: 56 - DNA, 16 bp, artificial
  primer 342r
ctgctgcsycccgtag

SEQ ID NO: 57 - DNA, 617 bp, E. coli
  AF056581_Pet
gaggtcaacaacctcaacaaacgtatgggtgatctgcgtgacattaacggtgaggccggtgcatgggcccgtatcatgagtggaaccgggtctgccggcggtggattcagtgacaactacacccacgttcaggtcggtgcggataacaaacatgaactcgatggccttgacctcttcaccggggtgaccatgacctataccgacagccatgcaggcagtgatgccttcagtggtgaaacgaagtctgtgggtgccggtctctatgcctctgccatgtttgagtccggagcatatatcgacctcatcggtaagtacgttcaccatgacaacgagtataccgcaactttcgccggccttggcaccagagactacagctcccactcctggtatgccggtgcggaagtcggttaccgttaccatgtaactgactctgcatggattgagccgcaggcggaacttgtttacggtgctgtatccgggaaacagttctcctggaaggaccagggaatgaacctcaccatgaaggataaggactttaatccgctgattgggcgtaccggtgttgatgtgggtaaatccttctccggtaaggactggaaagtcacagcccgcgccgg

SEQ ID NO: 58 - DNA, 617 bp, E. coli
  AF218073_Hbp
gaagttaacaacctgaacaaacgcatgggcgatttgagggatattaatggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcagacctgtacagcggtaaaacaaaatcatggggtggtggtttctatgccagtggtctgttccggtccggcgcttactttgatgtgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccgg

SEQ ID NO: 59 - DNA, 617 bp, E. coli
  AF297061_EspC1
gaggtcaataatctgaacaagcgtatgggtgacctgcgggatactcagggggatgccggcgtctgggcgcgcatcatgaacggtaccggttcggcagatggtggttacagcgataactacactcacgttcagattggtgccgacagaaagcatgagctggacggtgtggatttgttcacgggtgcattactgacctatacagacagcaatgcaagcagccacgccttcagtggtaaaaccaaatccgtggggggagggttgtacgcttcagcactctttgattccggggcttattttgacctgattggtaaatatctccatcacgacaatcagtacacggcgagttttgcgtctcttggtacaaaagactacagctctcattcctggtatgccggtgcagaggtcgggtatcgttaccacctgtcggaagagtcctgggtggagccacagatggagctggtttacggttctgtgtcaggaaaatcttttagctgggaagaccggggaatggccctgagcatgaaagacaaggattataacccactgattggccgtaccggtgttgacgtgggaagaaccttctccggagacgactggaaaattaccgcgcgagccgg

SEQ ID NO: 60 - DNA, 617 bp, E. coli
  AJ278144_EspI
gaggtcaacaacctgaataaacgtatgggtgacctgcgtgacatcaatggtgaagctggcgcatgggcacgtatcatgagcggaaccggctctgccggtggtggtttcagtgacaaccacacacatgttcaggtcggtgtcgacaaaaaacatgagctggacggactggatttgtttaccggcttcactgtcacacacactgacagcagtgcctctgctgatgctttcaaaggtaaaacaaaatctgtgggggccggactctatgcttccgccatgtttgattccggtgcctatatcgacctgattggtaagtatgttcatcatgataatgagtacaccgcaacctttgccggactcggaatccgtgattacagtacgcattcatggtatgccggtgctgaagcaggctaccgctgtcatgtcactgaggatacctggattgagccacaggcagaactggtttacggtgctgtatccggtaaacagtttgcatggaaggaccaggggatgcatctgtctatgaaggacagggactacaatccgctgattggtcgtaccggtgtggatgtgggtaaatccttctcaggtaaggactggaaagtgacagcccgtgccgg

SEQ ID NO: 61 - DNA, 617 bp, E. coli
  AJ586888_Sat
gaggtcaacaaccttaacaaacgtatgggtgatctgcgtgacattaacggtgagtccggtgcatgggcccgaatcatgagcggaaccgggtctgccggcggtggattcagtgacaactacacccacgttcaggtcggtgcggataacaaacatgaactcgatggccttgacctcttcaccggggtgaccatgacctataccgacagccatgcaggcagtgatgccttcagtggtgaaacgaagtctgtgggtgccggtctctatgcctctgccatgtttgagtccggagcatatatcgacctcatcggtaagtacgttcaccatgacaacgagtataccgcaactttcgccggccttggcaccagagactacagctcccactcctggtatgccggtgcggaagtcggttaccgttaccatgtaactgactctgcatggattgagccgcaggcggaacttgtttacggtgctgtatccgggaaacagttctcctggaaggaccagggaatgaacctcaccatgaaggataaggactttaatccgctgattgggcgtaccggtgttgatgtgggtaaatccttctcgggtaaggactggaaagtcacagcccgcgccgg

SEQ ID NO: 62 - DNA, 617 bp, E. coli
  AX276281_Pic_Ecoli
gaagtcaacaatctgaacaaacgtatgggtgacctgcgtgacacaaacggtgatgccggagcctgggcgcgcatcatgagtggtgccggttctgcagacggtggttacagtgataattacacccatgttcaggtcggctttgacaaaaaacatgaactggacggagtggacctgtttaccggtgtcacgatgacctataccgacagcagtgcagacagccatgcgttcagcggcaagacgaaatcggtggggggcggtctgtatgcttcagcattgtttgagtccggtgcctatatcgatttgattggtaaatatattcaccatgacaatgattacacaggtaactttgccggtctgggaacgaaacactacaacacccattcctggtatgccggtgctgaaacgggttaccgctatcacctgacagaggaaacgttcattgagccgcaggctgaactggtttacggcgccgtgtccgggaaaacattccgctggaaagacggtgatatggacctgagcatgaagaacagggacttcagtccgctgattggaagaacagggattgaactgggcaagaccttcagtggtaaggactggagtgtgacggcccgtgccgg

SEQ ID NO: 63 - DNA, 617 bp, E. coli
  AY163491_EatA
gaagtcaataatctgaacaaacgtatgggagatttacgggatagtcagggagatgctggagggtgggcgcgtatcatgaatggtaccggttcaggtgagagtggttacagagataactatacccacgttcagattggtgcagacagaaagcatgagctgaacggtatagatttattcaccggtgcattactgacttatacagacaacaatgctagcagccaggctttcagcggtaaaacaaaatcgctagggggaggggtgtatgcatcaggtctctttgagtctggagcttattttgacctgattggtaaatatctccatcatgataatcggtatacgttgaattttgcctccttgggggaaagaagctacacctcccattctttgtatgctggagctgaaatcgggtatcgttatcacatgtcagaaaatacatgggtggaaccacagatggaactggtttatggttcggtatcaggaaagtcatttaactggaaagaccagggaatgcaactgagtatgaaagacaaagactatcacccactaattggtcgaacaggtgtggatgtaggtagagcgttctctggagatacctggaaagtaacagtacgtgcagg

SEQ ID NO: 64 - DNA, 617 bp, E. coli
  AY258503_EpeA
gaagtcaacaacctgaacaaacgtatgggtgacctgcgtgacatcaatggcgaagccggtgcatgggcacgcatcatgagcggtactggctctgccagtggtggtttcagtgacaactacacgcacgttcaggtcggggtcgacaaaaaacatgagctggacggactggatttgtttaccggtttcactgtcacacacactgacagcagtgcctccgccgatgttttcagtggtaaaacgaagtctgtgggggctggcctgtatgcttccgccatgtttgattccggtgcctatatcgacctgattggcaagtatgttcaccatgataatgagtacactgcaacctttgccggactcggaacccgtgattacagcacgcattcatggtatgccggtgcagaagtgggctaccgctatcatgtcactgaggatgcctggattgagccacaggctgagctggtttacggttctgtatccggtaaacagtttgcatggaaggaccagggaatgcatctgtccatgaaggacaaggactacaatccgctgattggccgaactggtgtggatgtgggtaaatccttctctggtaaggactggaaagtgacagcccgtgccgg

SEQ ID NO: 65 - DNA, 617 bp, E. coli
  AY604009_SepA1
gaggtcaacaacctgaacaagcgtatgggtgacctgcgtgatacgcagggggatgccggtgtatgggcgcgtatcatgaacggtaccggttcggcagacggtggttacagcgataactacacgcacgttcagattggtgcagacagaaagcatgagctggatggtgtggatttgtttaccggtgcattgctgacctatacggacagtaatgcggacagtcatgcattcagtggtaaaacaaaatcagtgggtggtggtctgtatgcatctgcgctctttaattccggggcttattttgacctgattggtaaatatctccatcacgataatcagtacacggcgaattttgcctcactgggagaaaaagactacagctctcattcctggtatgccggtgctgaagtcgggtatcgttaccacctgtcggaagaatcctgggtggaacctcagatggagctggtttacggttctgtttcaggaaaatcctttaactgggaagaccggggaatggcactgaccatgaaagacaaggattataacccactgattggtcgtaccggtgttgacgtgggaagaagcttctccggagatgactggaaaatcacagcgcgcgccgg

SEQ ID NO: 66 - DNA, 617 bp, E. coli
  DD002707_Vat
gaagttaacaacctgaacaaacgcatgggcgatttgagggatattaacggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcaggcctgtacagcggtaaaacaaaatcatggggtggtggtttctatgccagtggtctgttccggtccggcgcttactttgatttgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccgg

SEQ ID NO: 67 - DNA, 617 bp, E. coli
  U69128_EspC2
gaggtcaataatctgaacaagcgtatgggtgacctgcgggatactcagggggatgccggcgtctgggcgcgcatcatgaacggtaccggttcggcagatggtggttacagcgataactacactcacgttcagattggtgccgacagaaagcatgagctggacggtgtggatttgttcacgggtgcattactgacctatacagacagcaatgcaagcagccacgccttcagtggtaaaaccaaatccgtggggggagggttgtacgcttcagcactctttgattccggggcttattttgacctgattggtaaatatctccatcacgacaatcagtacacggcgagttttgcgtctcttggtacaaaagactacagctctcattcctggtatgccggtgcagaggtcgggtatcgttaccacctgtcggaagagtcctgggtggagccacagatggagctggtttacggttctgtgtcaggaaaatcttttatctgggaagaccggggaatggccctgagcatgaaagacaaggattataacccactgattggccgtaccggtgttgacgtgggaagaaccttctccggagacgactggaaaattaccgcgcgagccgg

SEQ ID NO: 68 - DNA, 617 bp, E. coli
  X97542_EspP
gaggtcaacaacctgaacaaacgtatgggtgacctgcgtgatatcaacggcgaagccggtgcatgggcacgcatcatgagcggtaccggctctgccagtggtggtttcagtgacaactacacgcacgttcaggtcggggtcgacaaaaaacacgagctggacggactggatttgtttaccggtttcactgtcacacacactgacagcagtgcctccgccgatgttttcagtggtaaaacgaagtctgtgggggctggcctgtatgcttccgccatgtttgattccggtgcctatatcgacctgattggcaagtatgttcaccatgataatgagtacactgcaacctttgccggactcggaacccgtgattacagcacgcattcatggtatgccggtgcagaagcgggctaccgctatcatgtcactgaggatgcctggattgagccacaggctgagctggtttacggttctgtatccggtaaacagtttgcatggaaggaccagggaatgcatctgtccatgaaggacaaggactacaatccgctgattggccgaacgggtgtggatgtgggtaaatccttctctggtaaggactggaaagtgacagcccgtgccgg

SEQ ID NO: 69 - DNA, 617 bp, E. coli
  Y13614_PssA
gaggtcaacaacctgaacaaacgtatgggtgacctgcgtgatatcaacggcgaagccggtgcatgggcacgcatcatgagcggtaccggctctgccagtggtggtttcagtgacaactacacgcacgttcaggtcggggtcgacaaaaaacacgagctggacggactggatttgtttaccggtttcactgtcacacacactgacagcagtgcctccgccgatgttttcagtggtaaaacgaagtctgtgggggctggcctgtatgcttccgccatggttgattccggtgcctatatcgacctgattggcaagtatgttcaccatgataatgagtacactgcaacctttgccggactcggaacccgtgattacagcacgcattcatggtatgccggtgcagaagcgggctaccgctatcatgtcactgaggatgcctggattgagccacaggctgagctggtttacggttctgtatccggtaaacagtttgcatggaaggaccagggaatgcatctgtccatgaaggacaaggactacaatccgctgattggccgaacgggtgtggatgtgggtaaatccttctctggtaaggactggaaagtgacagcccgtgccgg

SEQ ID NO: 70 - DNA, 617 bp, E. coli
  NZ_AAJV01000028_Pic-like
gaagttaacaacctgaacaaacgtatgggtgaactgcgtgatacgaacggtgatgccggtgcctgggcccgtatcatgaacggcgcaggttcagccgatggcgggtacagtgataattacactcacgttcaggtcggctttgacaaaaaacatgtgctggatggcgtggacctgtttaccgggatcacaatgacctataccgacagcagtgcagacagtgatgcgttcagcgggaaaacaaaatccgtggggggcggtctgtatgcttcagcattgtttaactccggtgcctacattgatttgattggtaaatacattcaccataacaatgattacacaggcaactttgccggtctgggtacgaagcactacggaacccactcctggtatgccggagcggaaacgggttaccgttatcacctgacggaagacacatttattgagcctcaggccgaactggtttacggcgcagtgtccgggaaaacattccgctggaaagacggtgagatggacctgagtatgaagaacaaggatttcagcccgttgattggcagaacagggattgaactgggcaaaacattcagtggtaaggactggagtgtgacagcccgtgccgg

SEQ ID NO: 71 - DNA, 617 bp, E. coli
  Sat_AX702523
gaggtcaacaaccttaacaaacgtatgggtgatctgcgtgacattaacggtgagtccggtgcatgggcccgaatcattagcggaaccgggtctgccggcggtggattcagtgacaactacacccacgttcaggtcggtgcggataacaaacatgaactcgatggccttgacctcttcaccggggtgaccatgacctataccgacagccatgcaggcagtgatgccttcagtggtgaaacgaagtctgtgggtgccggtctctatgcctctgccatgtttgagtccggagcatatatcgacctcatcggtaagtacgttcaccatgacaacgagtataccgcaactttcgccggccttggcaccagagactacagctcccactcctggtatgccggtgcggaagtcggttaccgttaccatgtaactgactctgcatggattgagccgcaggcggaacttgtttacggtgctgtatccgggaaacagttctcctggaaggaccagggaatgaacctcaccatgaaggataaggactttaatccgctgattgggcgtaccggtgttgatgtgggtaaatccttctccggtaaggactggaaagtcacagcccgcgccgg

SEQ ID NO: 72 - DNA, 617 bp, Shigella
  AE005674_Pic_Shigella
gaagttaacaatctgaacaaacgtatgggtgacctgcgtgacacaaacggtgatgccggtgcctgggcgcgcatcatgagtggtgccggttctgcagacggtggttacagtgataattacacccatgttcaggtcggctttgacaaaaaacatgaactggacggtgtggacctgtttaccggtgtcacgatgacctataccgacagcagtgcagacagccatgcattcagcggaaagacgaaatcggtggggggcggtctgtatgcttcagcattgtttgagtccggtgcctatatcgatttgattggtaaatatattcaccatgacaatgattacacaggtaactttgctagcctgggaacgaaacactacaacacccattcctggtatgccggtgctgaaacgggttaccgctatcacctgacagaggacacgttcattgagccgcaggctgaactggtttacggcgccgtgtccgggaaaacattccgctggaaagacggtgatatggacctgagcatgaagaacagggacttcagtccgctggttggaagaacaggggttgaactgggcaagaccttcagtggtaaggactggagtgtgacggcccgtgccgg

SEQ ID NO: 73 - DNA, 617 bp, Salmonella bongori
  AY876285_Boa
gaagtcaacaacctgaacaagcgtatgggggatctgcgtgatactcagggcgaggacggaatgtgggtacgtatcatgaacggcgccggaaccggtgacgccggatattctgatcgttacacccatctgcaaacggggtttgataaaaaacaccggttgtcaggtgctgacttgttcactggtgtgttgatgagttataccgacagcagcgccagtggacgggcctacagcggcgacacgcattcgctcgggggtgggatgtacgcatccgtgatgtttgattcggggatatatatggatgttatcggcaagtatattcatcatgataatgactataacgccggttttgctggtctgggcaaacggaattacggtacacactcatggtatgctggcctggaaggcggataccgttaccgtctgacagaaagcctgtatattgagccgcaggcggaactggtatatggaaccgtctccggaacaacgctgaaatggaatgataatggtatggatgtgtcgatgcgcagcaaaacgtataatccgttgatagggcgtacaggcgtggcattgggcaaaacgttcagtggcaaggactggagcgttacggcccgtacagg

SEQ ID NO: 74 - DNA, 617 bp, citrobacter freundii
gaagttaacaacctgaacaaacgtatgggtgaactgcgtgatacgaacggtgatgccggtgcctgggcccgtatcatgaacggcgccgattcagcagagggcgggtacagtgataactacacccacgttcaggtcggttttgacaaaaaacatgcgctggacggtgtggacctgttcaccggtgtcacgatgacctataccgacagcagcgcagacagtgatgcgttcagcgggaagacaaaatccgtggggggcggcctgtatgcttcagcgctgtttaattccggcgcctatatcgatttgattggtaaatatattcaccatgacaatgattacacgggtaactttgccggcctggggacgaagcactacggaacccactcctggtatgccggagcggaaacgggttaccgttatcacctgacggaggacacctttattgagccgcaggccgaactggtttatggcgcggtgtccggcaaaacgttccgctggaaagacggtgatatggacctgagcatgaagaacaaggatttcagcccgttgattggcagaacagggattgagctgggtaaaaccttcagtggtaaggactggcgcgtgacggcccgggccgg

SEQ ID NO: 75 - DNA, 617 bp, Shigella sonnei
  CP000038_SigA
gaagtcaacaacctgaataaacgaatgggggatctgcgtgacaccaatggcgaggccggtgcatgggcccgcatcatgagcggagcaggttcagcttctagtggatacagtgacaactacacccatgtgcagattggtgtggataaaaaacatgagctggatggacttgaccttttcactggtctgactatgacgtataccgacagtcatgccagcagtaatgcattcagtggcaagacgaagtccgtcggggcaggtctgtatgcttccgctatatttgactctggtgcctatatcgacctgattagtaagtatgttcaccatgataatgagtactcggcgacctttgctggactcggaacaaaagactacagttctcattccttgtatgtgggtgctgaagcaggctaccgctatcatgtaacagaagactcctggattgagccgcaggcagaactggtttatggggccgtatcaggtaaacggttcgactggcaggatcgcggaatgagcgtgaccatgaaggataaggactttaatccgctgattgggcgtaccggtgttgatgtgggtaaatccttctccggtaaggactggaaagtcacagcccgcgccgg

SEQ ID NO: 76 - DNA, 617 bp, Shigella flexneri
  Z48219_SepA
gaggtcaataacctgaacaaacgtatgggtgacctgcgggatactcagggggatgccggtgtctgggcacgcataatgaatggtaccggttcggcagatggtgactacagcgataactacactcacgttcagattggtgtcgacagaaagcatgagctggacggtgtggatttatttacgggggcattgctgacctatacggacagcaatgcaagcagccacgcattcagtggaaaaaacaaatccgtgggtggcggtctgtatgcctctgcactctttaattccggagcttattttgacctgattggtaaatatctccatcatgataatcagcacacggcgaattttgcctcactgggaacaaaagactacagctctcattcctggtatgccggtgctgaagttggttatcgttaccacctgacgaaagagtcctgggtggagccacagatagagctggtttacggttctgtatcaggaaaagcttttagctgggaagcccggggaatggctctgagcatgaaagacaaggattataacccactgattggccgtactggtgttgacgtgggaagagccttctccggagacgactggaaaatcacagctcgagccgg

SEQ ID NO: 77 - DNA, 617 bp, Citr_rodendium; identical to SEQ ID NO: 74

SEQ ID NO: 78 - DNA, 617 bp, Shigella flexneri
  SepA_AF348706
gaggtcaataacctgaacaaacgtatgggtgacctgcgggatactcagggggatgccggtgtctgggcacgcataatgaatggtaccggttcggcagatggtgactacagcgataactacactcacgttcagattggtgtcgacagaaagcatgagctggacggtgtggatttatttacgggggcattgctgacctatacggacagcaatgcaagcagccacgcattcagtggaaaaaacaaatccgtgggtggcggtctgtatgcctctgcactctttaattccggagcttattttgacctgattggtaaatatctccatcatgataatcagcacacggcgaattttgcctcactgggaacaaaagactacagctctcattcctggtatgccggtgctgaagttggttatcgttaccacctgacgaaagagtcctgggtggagccacagatagagctggtttacggttctgtatcaggaaaagcttttagctgggaagaccggggaatggctctgagcatgaaagacaaggattataacccactgattggccgtactggtgttgacgtgggaagagccttctccggagacgactggaaaatcacagctcgagccgg

SEQ ID NO: 79 - DNA, 617 bp, Shigella flexneri; identical to SEQ ID NO: 76
  SepA_Z48219

SEQ ID NO: 80 - DNA, 3120 bp, E. coli
  CDS (1)..(3120)
  Ag43 U24429
atgaaccgacatttgaatacctgctacaggctggtatggaatcacatgacgggcgcttttgtggttgcctccgaattggcccgcgcacggggtaaacgtggcggtgtggcggttgcactgttttttgccgcagtcaagtcactcccggtgctggctgctgacattgttgtgcacccgggagaaaccgtgaacggcggaacactggcaaatcatgacaaccagattgtcttcggtacgaccaacggaatgaccatcagtaccgggctggagtatgggccggataacgaggccaataccggcgggcaatgggtacaggatggcggaacagccaacaaaacgactgtcaccagtggtggtcttcagagagtgaaccccggtggaagtgtctcagacacggttatcagtgccggaggcggacagagccttcagggacgggctgtgaacaccacgctgaatggtggcgaacagttgatgcatgagggggcgatagccacgggaaccgtcattaatgataagggctggcaggtcgtcaagcccggtacagtggcaacggataccgtttttaataccggggcggaagggggaccggatgcagaaaacggtgataccgggcagtttgttcgcggggatgccgtacgcacaaccatcaataaaaacggtcgccagattgtgagagctgaaggaacggcaaataccactgtggtttatgccggcggcgaccagactgtacatggtcacgcactggataccacgctgaatgggggataccagtatgtgcacaacggcggtacagcgtctgacactgttgtgaacagtgacggctggcagattgtcaaaaacgggggtgtggccgggaataccaccgttaatcagaagggcagactgcaggtggacgccggtggtacatccacgattgtcaccctgaagcaggggggcgcattggttaccagtacggctacaaccgttaccggcataaaccgcctgggagcattctctgttgtggagggtaaagctgataatgtcgtactggaaaatggcggacgcctggatgtgctgaccggacacacagccactaatacccgcgtggatgatggcggaacgctggatgtccggcaaggcggcaccgccaccaccgtatccatgggaaatggcggtgtactgctggccgattccggtgccgctgtcagtggtacccggagcgacggaaaggcattcagtatcggaggcggtcaggcggatgccctgatgctggaaaaaggcagttcattcacgctgaacgccggtgatacggccacggataccacggtaaatggcggactgttcaccgccaggggcggcacactggcgggcaccaccacgctgaataacggcgccatacttaccctttccgggaagacggtgaacaacgataccctgaccatccgtgaaggcgatgcactcctgcagggaggctctctcaccggtaacggcagcgtggtaaaatcaggaaatggcactctcactgtcagcaacaccacactcacccagaaagccgtcaacctgaatgaaggcacgctgacgctgaacgacagtaccgtcaccacggatgtcattgctcagcgcggtacagccctgaagctgaccggcagcactgtgctgaacggtgccattgaccccacgaatgtcactctcgcctccggggccacctggaatatccccgataacgccacggtgcagtcggtggtggatgacctcagccatgccggacagatttatttcacctccacccgcacagggaagtttgtaccggcaaccctgaaagtgaaaaacctgaacggacagaatggcaccatcagcctgcgtgtacgcccggatatggcacagaacaatgctgacagactggtcattgacggcggcagggcaaccggaaaaaccatcctgaacctggtgaacgccggcaacagtgcgtcggggctggcgaccagcggtaagggtattcaggtggttgaagccattaacggtgccaccacggaggaaggggcctttgttcaggggaacaggctgcaggccggtgcctttaactactccctcaaccgggacagtgatgagagctggtatttgcgcagtgaaaatgcttatcgtgcaaaagtccccctgtatgcctccatgctgacacaggcaacggactatgaccggattgtggcaggctcccgcagccatcagaccggtgtaaatggtgaaaacaacagcgtccgtctcagcattcagggcagtcatttcggtcacgataacaatggcggtattgcccgtggggccacgccggaaagcagcggcagctatggatttgtccgtctggagggtgacctgatgagaacagaggttgccggtatgtctgtgaccgcgggggtatatggtgctgctggccatccttccgttgatgttaaggatgatgacggctcccgtgtcggcacggtccgggatgatgccggcagcctgggcggatacatgaatctgatatacaacgcctccggactgtgggctgacattgtggccctgggagcccgccacagcatgaaagcgtcaacggacaataacgacttccgcgcccggggctggggctggctgggctcactggaaaccggtctgcccttcagtatcactgacaacctgatgctggagccactactgcagtatacctggcagggactttccctggatgacggtaaggacaacgccggttatgtgaagttcgggcatggcagtgcacaacatgtgcgtgccggtttccgtctgggcagccacaacgatatgacctttggcgaaggcacctcatcccgcgcccccctgcgtgacagtgcaaaacacagtgtgagtgagttaccggtgaactggtgggtacagccttctgttatccgcaccttcagctcccggggagatatgcgtgtggggacttccactgcaggcagcgggatgacgttttctccctcacagaatggcacatcactggacttgcaggccggactggaagcccgtgtccgggaaaatatcaccctgggcgttcaggccggttatgcccacagcgtcagcggcagcatcgctgaagggtataacggtcaggccacactgaatgtgaccttctga

SEQ ID NO: 81 - protein, 1039 aa, E. coli
  amino acids 718-822 are encoded by the nucleotide sequence shown in Figure 5, in the +3 reading frame
MNRHLNTCYRLVWNHMTGAFVVASELARARGKRGGVAVALFFAAVKSLPVLAADIVVHPGETVNGGTLANHDNQIVFGTTNGMTISTGLEYGPDNEANTGGQWVQDGGTANKTTVTSGGLQRVNPGGSVSDTVISAGGGQSLQGRAVNTTLNGGEQLMHEGAIATGTVINDKGWQVVKPGTVATDTVFNTGAEGGPDAENGDTGQFVRGDAVRTTINKNGRQIVRAEGTANTTVVYAGGDQTVHGHALDTTLNGGYQYVHNGGTASDTVVNSDGWQIVKNGGVAGNTTVNQKGRLQVDAGGTSTIVTLKQGGALVTSTATTVTGINRLGAFSVVEGKADNVVLENGGRLDVLTGHTATNTRVDDGGTLDVRQGGTATTVSMGNGGVLLADSGAAVSGTRSDGKAFSIGGGQADALMLEKGSSFTLNAGDTATDTTVNGGLFTARGGTLAGTTTLNNGAILTLSGKTVNNDTLTIREGDALLQGGSLTGNGSVVKSGNGTLTVSNTTLTQKAVNLNEGTLTLNDSTVTTDVIAQRGTALKLTGSTVLNGAIDPTNVTLASGATWNIPDNATVQSVVDDLSHAGQIYFTSTRTGKFVPATLKVKNLNGQNGTISLRVRPDMAQNNADRLVIDGGRATGKTILNLVNAGNSASGLATSGKGIQVVEAINGATTEEGAFVQGNRLQAGAFNYSLNRDSDESWYLRSENAYRAKVPLYASMLTQATDYDRIVAGSRSHQTGVNGENNSVRLSIQGSHFGHDNNGGIARGATPESSGSYGFVRLEGDLMRTEVAGMSVTAGVYGAAGHPSVDVKDDDGSRVGTVRDDAGSLGGYMNLIYNASGLWADIVALGARHSMKASTDNNDFRARGWGWLGSLETGLPFSITDNLMLEPLLQYTWQGLSLDDGKDNAGYVKFGHGSAQHVRAGFRLGSHNDMTFGEGTSSRAPLRDSAKHSVSELPVNWWVQPSVIRTFSSRGDMRVGTSTAGSGMTFSPSQNGTSLDLQAGLEARVRENITLGVQAGYAHSVSGSIAEGYNGQATLNVTF

SEQ ID NO: 82 - DNA, 317 bp, E. coli
  AE005174
tgacacaggcaatggactatgaccggattctggcaggctcccgcagccatcagaccggtgtaaacggtgaaaataacagcgtccgtctcagcattcagggcggtcatctcggtcacgataacaacggcggtattgcccgtggagccacgccggaaagcagcggcagctatggcttcgtccgtctggagggtgacctgctcagaacagaggttgccggtatgtctctgacgacaggggtgtatggtgctgcaggccattcttccgttgatgttaaggatgatgacggttcccgcgccggcacggtccgggatgatgcc

SEQ ID NO: 83 - DNA, 317 bp, E. coli
  AE014075
tgacacaggcaatggactatgaccggattctggcaggctcacgcagccatcagaccggtgtaaacggtgaaaataacagcgtccgtctcagcattcagggcggccatctcggtcacgataacaacggcggtattgcccgtggggccacgccggaaagcagcggcagctatggcttcgtccgcctggagggtgacctgctgagaacagatgttgccggtatgtctgtgaccgcagggatatatggtgctgcaggccattcttccgttgatgttaaggatgatgacggctcccgtgccggcacggtccgggatgatgcc

SEQ ID NO: 84 - DNA, 317 bp, E. coli
  AF233271
tgacacaggcaatggactatgaccggattgtggcaggctcccgcagccatcagaccggtgtaaatggtgaaaacaacagcgtccgtctcagcattcagggcggtcatctcggtcacgataacaatggcggtattgcccgtggggccacgccggaaagcagcggcagctatggattcgtccgtctggagggtgacctgatgagaacagaggttgccggtatgtctgtgaccgcgggggtatatggtgctgctggccattcttccgttgatgttaaggatgatgacggctcccgtgtcggcacggtccgggatgatgcc

SEQ ID NO: 85 - DNA, 317 bp, E. coli
  AF233272
tgacacaggcaatggactatgaccggattgtggcaggctcccgcagccatcagaccggtgtaaatggtgaaaacaacagcgtccgtctcagcattcagggcggtcatctcggtcacgataacaatggcggtattgcccgtggggccacgccggaaagcagcggcagctatggattcctccgtctggagggtgacctgatgagaacagaggttgccggtatgtctgtgaccgcgggggtatatggtgctgctggccattcttccgttgatgttaaggatgatgacggctcccgtgtcggcacggtccgggatgatgcc

SEQ ID NO: 86 - DNA, 317 bp, E. coli
  AJ303141
tgacacaggcaatggactatgaccggattctggcaggctcccgcagccatcagaccggtgtaaacggtgaaaataacagcgtccgtctcagcattcagggcggtcatctcggtcacgataacaacggcggtattgcccgtggagccacgccggaaagcagcggcagctatggcttcgtccgtctggagggtgacctgctcagaacagaggttgccggtatgtctgtgaccgcgggggtatatggtgctgctggccattcttccgttgatgttaaggatgatgacggctcccgtgccggcacggtccgggatgatgcc

SEQ ID NO: 87 - DNA, 317 bp, E. coli
  AJ586887
tgacacaggcaatggactatgaccggattctggcaggctcccgcagccatcagaccggtgtaaacggtgaaaataacagcgtccgtctcagcattcagggcggccatctcggtcacgataacaacggcggtattgcccgtggggccacgccggaaagcagcggcagctatggcttcgtccgtctggagggtgacctgctcagaacagaggtcgccggtatgtcactgacgacaggagtgtatggtgctgcaggccattcttccgttgatgttaaggatgatgacggctcccgcgccggcacggtccgggatgatgcc

SEQ ID NO: 88 - DNA, 317 bp, E. coli
  AJ586888
tgacacaggcaatggactatgaccggattctggcaggctcccgcagccatcagaccggtgtaaacggtgaaaataacagcgtccgtctcagcattcagggcggccatctcggtcacgataacaacggcggtattgtccgtggtgccacgccggaaagcagcggcagctatggcttcgtccgtctggagggtgacctgctcagaacagaggttgccggtatgtcactgacgacaggagtgtatggtgctgcaggccattcttccgttgatgttaaggatgatgacggctcccgtgccggcacggtccgggatgatgcc

SEQ ID NO: 89 - DNA, 317 bp, E. coli
  AJ617685
tgacacaggcaatggactatgaccggattctggcaggctcccgcagccatcagaccggtgtaaatggtgaaaacaacagcttccgtctcagcattcagggcggtcatctcgggcacgttaacaacggtggtattgcccgtggggccacgccggaaagcagcggcagctatggcctcgtccgtctggagggtgacctgctgagaacagaggttgccggtatgtcactgacgacaggagtgtatggtgctgcaggccattcttccgttgatgttaaggatgatgacggctcccgtgccggcacggtccgggatgatgcc

SEQ ID NO: 90 - DNA, 317 bp, E. coli
  AP009048
tgacacaggcaatggactatgaccggattgtggcaggctcccgcagccatcagaccggtgtaaatggtgaaaacaacagcgtccgtctcagcattcagggcggtcatctcggtcacgataacaatggcggtattgcccgtggggccacgccggaaagcagcggcagctatggattcgtccgtctggagggtgacctgatgagaacagaggttgccggtatgtctgtgaccgcgggggtatatggtgctgctggccattcttccgttgatgttaaggatgatgacggctcccgtgccggcacggtccgggatgatgcc

SEQ ID NO: 91 - DNA, 317 bp, E. coli
  AR580480
tgacacaggcaatggactatgaccggattctggcaggctcccgcagccatcagaccggtgtaagcggtgaaaataacagcgtccgtctcagcattcagggcggtcatctcgggcacgataacaacggtggtattgcccgtggggccacgccggaaagcagcggcagctatggcttcgtccgtctggagggtgacctgctcagaacagaggttgccggtatgtctgtgaccgcgggggtatatggtgctgctggccattcttccgttgatgttaaggattatgacggttcccgcgccggcacggtccgggatgatgcc

SEQ ID NO: 92 - DNA, 317 bp, E. coli; identical to SEQ ID NO: 90
  AX370193

SEQ ID NO: 93 - DNA, 317 bp, E. coli; identical to SEQ ID NO: 87
  AX702425

SEQ ID NO: 94 - DNA, 317 bp, E. coli; identical to SEQ ID NO: 83
  AX702524

SEQ ID NO: 95 - DNA, 317 bp, E. coli
  AY857617
tgacacaggcaatggactatgaccggattctggcaggctcccgcagccatcagaccggtgtaagcggtgaaaataacagcgtccgtctcagcattcagggcggtcatctcggtcacgataacaacggcggtattgcccgtggagccacgccggaaagcagcggcagctatggcttcgtccgtctggagggtgacctgctcagaacagaggttgccggtatgtctgtgaccgcgggggtatatggtgctgctggccattcttccgttgatgttaaggatgatgacggctcccgtgccggcacggtccgggatgatgcc

SEQ ID NO: 96 - DNA, 317 bp, E. coli; identical to SEQ ID NO: 82
  BA000007

SEQ ID NO: 97 - DNA, 317 bp, E. coli; identical to SEQ ID NO: 82
  BD184766

SEQ ID NO: 98 - DNA, 317 bp, E. coli; identical to SEQ ID NO: 91
  BD195283

SEQ ID NO: 99 - DNA, 317 bp, E. coli; identical to SEQ ID NO: 90
  BD444174

SEQ ID NO: 100 - DNA, 317 bp, E. coli
  CP000243
tgacacaggcaatggactatgaccggattctggcaggctcccgcagccatcagagcggtgtaagcggtgaaaataacagcgtccgtctcagcattcagggcggtcatctcgggcacgataacaacggtggtattgcccgtggggccacgccggaaagcaacggcagctatggcttcgtccgtctggagggtgacctgctcagaacagaggttgccggtatgtcactgacgacaggagtgtatggtgctgcaggccattcttccgttgatgttaaggatgatgacggctcccgcgccggcacggtccgggatgatgcc

SEQ ID NO: 101 - DNA, 317 bp, E. coli; identical to SEQ ID NO: 82
  CS148067

SEQ ID NO: 102 - DNA, 317 bp, E. coli; identical to SEQ ID NO: 90
  U00096

SEQ ID NO: 103 - DNA, 317 bp, E. coli
  U24429
tgacacaggcaacggactatgaccggattgtggcaggctcccgcagccatcagaccggtgtaaatggtgaaaacaacagcgtccgtctcagcattcagggcagtcatttcggtcacgataacaatggcggtattgcccgtggggccacgccggaaagcagcggcagctatggatttgtccgtctggagggtgacctgatgagaacagaggttgccggtatgtctgtgaccgcgggggtatatggtgctgctggccatccttccgttgatgttaaggatgatgacggctcccgtgtcggcacggtccgggatgatgcc

SEQ ID NO: 104 - DNA, 317 bp, E. coli
  X16664
tgacacaggcaatggactatgaccggattctggcaggctcccgcagccatcagaccggtgtaaacgttaaaaataacagcgtccgtctcagcattcagggcggtcatctcggtcacgataacaacggcggtattgcccgtggggccacgccggaaagcagcggcagctatggcttcgtccgtctggagggggacctgctgagaacagaggttgccggtatgtctgtgaccgcgggggtatatggcgctgcaggccattcttccgttgatgttaaggatgatgacggctcccgtgccggcacggtccgggatgatgcc

SEQ ID NO: 105 - DNA, 317 bp, Shigella dysenteriae
  CP000034
tgacacaggcaatggactatgaccggattctggcaggctcccgcagccatcagaccagtgtaagcggtgaaaataacagcgtccgtctcagcattcagggcggtcatctcggacacgataacaacggtggtattgcccgtggggccacgccggaaagcagcggcagctatggcctcgtccgtctggagggtgacctgctgagaacagaggttgccggtatgtcactgacgacaggagtgtatggtgctgcaggccattcttccgttgatgttaaggatgatgacggttcccgcgccggcacggtccgggatgatgcc

SEQ ID NO: 106 - DNA, 317 bp, Shigella flexneri
  AF326777
tgacacaggcaatggactatgaccggattctggcaggctcccgcagccatcagaccggtgtaaacggtgaaaataacagcgtccgtctcagcattcaggacggtcatctcggtcacgataacaacggcggtattgcccgtggagccacgccggaaagcagcggcagctatggcttcgtccgtctggagggtgacctgctgagaacagaggttgccggtatgtctctcacgacaggggtgtatggtgccgcaggccattcttccgttgatgttaagaatgatgacggttcccgcgccggcacggtccgggatgatgcc

SEQ ID NO: 107 - DNA, 317 bp, unknown; identical to SEQ ID NO: 82
  Unknown species

SEQ ID NO: 108 - DNA, 317 bp, Unknown; identical to SEQ ID NO: 82
  Unknown species

SEQ ID NO: 109 - DNA, 317 bp, Shigella flexneri; identical to SEQ ID NO: 95
  AE005674

SEQ ID NO: 110 - DNA, 317 bp, Shigella flexneri; identical to SEQ ID NO: 95
  AE014073

SEQ ID NO: 111 - DNA, 317 bp, Shigella flexneri; identical to SEQ ID NO: 95
  AF200692

SEQ ID NO: 112 - DNA, 317 bp, Shigella flexneri; identical to SEQ ID NO: 95
  CS148243

(19) United States
(12) Patent Application Publication
(10) Pub. No.: US 2009/0305267 A1
(43) Pub. Date: Dec. 10, 2009

(54) MICROBIAL MARKERS OF INFLAMMATORY BOWEL DISEASE

(75) Inventors: Denis O. Krause, Winnipeg (CA); Charles N. Bernstein, Winnipeg (CA)

Correspondence Address: SMART & BIGGAR;P.O. BOX 2999, STATION D, 900-55 METCALFE STREET, OTTAWA, ON, K1P 5Y6, CA

(73) Assignee: UNIVERSITY OF MANITOBA, Winnipeg, MB (CA)

(21) Appl. No.: 12/299,397

(22) PCT Filed: May 3, 2007
(86) PCT No.: PCT/CA2007/000740

§ 371 Date: Jul. 8, 2009

Related U.S. Application Data
(60) Provisional application No. 60/796,879, filed on May 3, 2006.

(51) Int. Cl. C12Q 1/68; G01N 33/569

(52) U.S. Cl. 435/6; 435/7.32

(57) ABSTRACT

The present invention relates to bacterial serine protease autotransporter (SPATE) and Antigen 43 (Ag43) and their various uses relating to inflammatory bowel disease (IBD), specifically in the diagnosis of IBD and the screening of potential agent for treating IBD. The invention also relates to methods for cultivating and identifying enteric microbes.

### REFERENCE TO RELATED APPLICATIONS

This application claims the benefit of U.S. Provisional Application No. 60/796,879, filed May 3, 2006, the content of which is herein incorporated by reference.

### FIELD OF INVENTION

The present invention relates to microbial genes of the autotransporter family, specifically the serine protease autotransporter (SPATE) family and the Antigen 43 (Ag 43) family, and their role in inflammatory bowel disease (IBD). The invention also relates to methods for cultivating and identifying microbes of the gut.

### BACKGROUND OF THE INVENTION

Inflammatory bowel disease (IBD) is a collective term for ulcerative colitis (UC) and Crohn's disease (CD). These diseases are chronic inflammatory diseases of the digestive tract, potentially leading to severe inflammation, ulceration, and obstruction, the end-point of which may be surgical resection.

IBD is thought to be a result of recognition of a microbial antigen(s) by a dysfunctional immune system in a genetically predisposed host. Many bacteria have been linked to IBD,but specific bacteria may have been missed because not more than 30% of the microbial diversity in the gut can be cultured (refs 8 to 12).

Several bacteria have been implicated in the aetiology of IBD, the most prominent among these being Mycobacterium paratuberculosis. Other bacteria that have been associated are members of the Enterobacteriaceae, Helicobacter pylori, and Bacteroides species.

### SUMMARY OF THE INVENTION

We describe here an invention based in part, but is not limited to, the use of culture-independent surveys of microbial diversity as a prelude to targeted cultivation of bacteria. As an example, we describe the use of ribosomal intergenic spacer analysis (RISA) of biopsy tissue to identify nucleic acids that are consistently associated with IBD. From our survey, we found that genes of the microbial serine protease autotransporter (SPATE) family and the Ag43 family are useful markers for diagnosing and prognosing IBD.

We describe a method for diagnosing inflammatory bowel disease (IBD) or determining susceptibility to developing IBD in a subject. The method comprises the step of assaying for SPATE or Ag43 or both in an enteric bacteria-containing sample from the subject, wherein the presence of SPATE or Ag43 or both in the sample indicates that the subject has IBD or is susceptible to developing IED.

The subject undergoing diagnosis or prognosis for IBD includes those having symptoms of IBD, or are suspected of having IBD. IBD includes ulcerative colitis (UC) and Crohn's disease (CD).

In the diagnostic or prognostic method, the SPATE or Ag43 being assayed may be SPATE nucleic acid or SPATE polypeptide or Ag43 nucleic acid or Ag43 polypeptide. With respect to detecting SPATE or Ag43 nucleic acid, the method may comprise detecting a region of SPATE or a region of Ag43 which is conserved among enteric bacteria, or detecting a region of SPATE or a region of Ag43 which is conserved among enteric E. coli, or detecting a region of SPATE or a region of Ag43 which is specific to enteric E. coli, or detecting a region of SPATE or a region of Ag43 which is conserved among virulent enteric strains of E. coli, or detecting a region of SPATE or a region of Ag43 which is specific to virulent enteric strains of E. coli, or detecting a region of SPATE or a region of Ag43 which is specific to E. coli of the B2 or D or the B2+D genotype. Polymerase chain reaction (PCR) may be used to detect SPATE or Ag43 nucleic acid. Primer sequences may be designed for PCR amplification of the target sequences in these methods.

In the diagnostic or prognostic method, the SPATE or Ag43 being assayed may be SPATE or Ag43 polypeptide. The method may employ an immunoassay for SPATE or Ag43 polypeptide. For example, the immunoassay may employ an antibody which is immunospecific against SPATE or Ag43 of enteric bacteria, or is immunospecific against SPATE or Ag43 of enteric E. coli, or is immunospecific against SPATE or Ag43 of virulent enteric strains of E. coli, or is immunospecific against SPATE or Ag43 of E. coli of the B2 or D or B2+D genotype. The diagnostic or prognostic method may involve determining the level of SPATE or Ag43 in the sample, in which case a higher level of SPATE or Ag43 compared to a cut-off value would indicate that the subject has IBD or or is susceptible to developing IBD.

In the various methods disclosed here which include diagnostic or prognostic methods, the subject includes human or an animal suitable for use in an IBD disease model, e.g. mouse.

In the various methods disclosed here which include diagnostic or prognostic methods, the sample is any sample containing enteric bacteria. Sources for samples include gut tissue biopsy, intestinal mucosa, stool or fecal matter, or intestinal wash. In particular, the sample may be a colonoscopy tissue biopsy from the lower gastrointestinal (GI) tract.

We also describe a method for evaluating effectiveness of a treatment for inflammatory bowel disease (IBD) in a subject, the method comprising the steps of: (i) determining serine protease autotransporter (SPATE) level or Ag43 level, or both, in an enteric bacteria-containing sample from a treated subject having IBD, and (ii) comparing the SPATE level or the Ag43 level or both from step (i) to SPATE and Ag43 levels determined in an enteric bacteria-containing sample from an untreated subject having IBD. The untreated and treated subjects are either different subjects or are the same subjects before and after undergoing the treatment. A lower level of SPATE or Ag43 or both in treated subjects indicates that the treatment is effective for IBD.

The subject undergoing evaluation for IBD treatment includes those having ulcerative colitis (UC) or Crohn's disease (CD).

In the methods for evaluating IBD treatment, the SPATE or Ag43 level may be determined by assaying for SPATE nucleic acid or SPATE polypeptide or Ag43 nucleic acid or Ag43 polypeptide. With respect to detecting nucleic acid, the method may comprise detecting a region of SPATE or Ag43 which is conserved among enteric bacteria, or detecting a region of SPATE or Ag43 which is conserved among enteric E. coli, or detecting a region of SPATE or Ag43 which is specific to enteric E. coli, or detecting a region of SPATE or Ag43 which is conserved among virulent enteric strains of E. coli, or detecting a region of SPATE or Ag43 which is specific to virulent enteric strains of E. coli, or detecting a region of SPATE or Ag43 which is specific to E. coli of the B2 or D or the B2+D genotype. Polymerase chain reaction (PCR) may be used to detect SPATE or Ag43 nucleic acid. Primer sequences may be designed for PCR amplification of the target sequences in these methods.

In the methods for evaluating IBD treatment, the SPATE or Ag43 being assayed may be SPATE or Ag43 polypeptide. The method may employ an immunoassay for SPATE or Ag43 polypeptide. For example, the immunoassay may employ an antibody which is immunospecific against SPATE or Ag43 of enteric bacteria, or is immunospecific against SPATE or Ag43 of enteric E. coli, or is immunospecific against SPATE or Ag43 of virulent enteric strains of E. coli, or is immunospecific against SPATE of E. coli of the B2 or D or the B2+D genotype. The immunoassay comprises the steps of: (i) contacting an enteric bacteria-containing sample from a subject with an antibody immunospecific against SPATE or an antibody immunospecific against Ag43 or both antibodies, under conditions suitable to form a complex between SPATE or Ag43 and the antibody; and (ii) detecting presence or absence of the complex. Presence of the complex indicates that the subject has or is susceptible to developing IBD.

The diagnostic or prognostic method may involve determining the level of SPATE or Ag43 in the sample, in which case a higher level of SPATE or Ag43 compared to a cut-off value would indicate that the subject has IBD or or is susceptible to developing IBD.

We also describe a commercial package for diagnosing IBD or determining susceptibility to developing IBD in a subject. The subject includes those having symptoms of IBD, or are suspected of having IBD, including UC or CD. The package comprises an agent for detecting serine protease autotransporter (SPATE), or an agent for detecting Ag43 in an enteric bacteria-containing sample from the subject, and instructions for using the agent(s) to detect SPATE or Ag43 in the sample. The kit is therefore made to be used for diagnosing or prognosing IBD in the subject.

The agent, which is part of the commercial package, includes those agents useful for detecting SPATE or Ag43 nucleic acid. Such agents include those that detect a region of SPATE or a region or Ag43 which is conserved among enteric bacteria, a region of SPATE or a region or Ag43 which is conserved among enteric E. coli, a region of SPATE or a region or Ag43 which is specific to enteric E. coli, a region of SPATE or a region or Ag43 which is conserved among virulent enteric strains of E. coli, a region of SPATE or a region or Ag43 which is specific to virulent enteric strains of E. coli, or a region of SPATE or a region or Ag43 which is specific to E. coli of the B2 or D or the B2+D genotype. The agent may be those used to detect SPATE or Ag43 nucleic acid by polymerase chain reaction (PCR). In particular, the agent may comprise primer sequences designed to amplify and detect the target sequences.

The agent, which is part of the commercial package, also includes those agents useful for detecting SPATE or Ag43 polypeptide. For example, the agent may be for detecting SPATE or Ag43 polypeptide in an immunoassay. The agent may be an antibody which is immunospecific against SPATE or Ag43 of enteric bacteria, or against SPATE or Ag43 of enteric E. coli, or against SPATE or Ag43 of virulent enteric strains of E. coli, or against SPATE or Ag43 of E. coli of the B2 or D or the B2+D genotype. The commercial package may further comprise SPATE polypeptide or Ag43 polypeptide for generating a standard curve(s), as a standard against which the test level of SPATE polypeptide or Ag43 polypeptide is assessed as being statistically higher, lower, or equivalent, to normal.

We also describe a method of testing potential therapeutic agents for treating inflammatory bowel disease (IBD), the method comprising contacting a test compound with SPATE polypeptide or with Ag43 polypeptide and determining whether the test compound binds to SPATE or inhibits SPATE protease activity, or whether the test compound binds Ag43 or inhibits Ag43 aggregation activity or cell adhesion activity, wherein binding to SPATE or inhibition of SPATE protease activity, or binding to Ag43 or inhibition of Ag43 aggregation or cell adhesion indicates that the compound is a potential therapeutic agent for treating IBD.

We also describe a method for cultivating enteric microbes. The method comprises the steps of: a) resuscitating an enteric bacteria-containing sample from a subject, including those having IBD or specifically UC or CD, by resuspension in buffered peptone water; and b) culturing the resuspension. The enteric bacteria being cultivated may be a wide range of microbes including bacteria, or specific microbes such as E. coli. The enteric bacteria-containing sample from which enteric microbes are cultivated may be a colonoscopy tissue biopsy. In some embodiments, there is no need for washing the tissue or removing mucus before resuscitating the microbes in the sample.

We also describe a method for phylogenetic identification of enteric bacteria associated with inflammatory bowel disease (IBD). The method comprises the steps of: (i) amplifying DNA from an enteric bacteria-containing sample from a subject having IBD; (ii) amplifying DNA from an enteric bacteria-containing sample from a subject free of IBD; (iii) comparing the amplified DNA obtained from step (i) with amplified DNA obtained from step (ii) to mark out DNA that is associated with the subject having IBD; and (iv) performing phylogenetic analysis to identify bacteria containing the DNA associated with the subject having IBD. In this method, the enteric bacteria-containing sample may be a colonoscopy tissue biopsy. The DNA amplification may comprise PCR amplification of a polymorphic region flanked by conserved sequences of enteric bacteria, in which the PCR amplification employs primers specific for the conserved sequences. For example, the primers may be specific to at least a portion of the 16S rRNA gene, or to at least a portion of the 23S rRNA gene. The primers may be specific to at least a portion of both the 16S and the 23S rRNA genes.

We also describe a method for phylogenetic identification of enteric bacteria associated with inflammatory bowel disease (IBD), but using DNA from cultivated bacteria. The method comprises the steps of: (i) amplifying DNA from enteric bacteria wherein the bacteria are from a subject having IBD and were cultivated according to the method described above; (ii) amplifying DNA from enteric bacteria wherein the bacteria are from an IBD-free subject and were cultivated according to the method described above; (iii) comparing the amplified DNA obtained from step (i) with amplified DNA obtained from step (ii) to mark out DNA from bacteria associated with the subject having IBD; and (iv) performing phylogenetic analysis to identify the bacteria associated with the subject having IBD.

### BRIEF DESCRIPTION OF THE DRAWINGS

FIG. 1 shows a RISA analysis of biopsy samples from IBD and control tissues.

FIG. 2 shows the proportions of E. coli and non-E. coli in biopsy tissues from controls, and UC and CD patients.

FIG. 3A-D shows an alignment of SPATE nucleotide sequences (SEQ ID NOs:57-79) amplified using RISA on biopsy tissue.

FIG. 4A-D shows an alignment of pic-like gene with other SPATE (SEQ ID NOs:1 and 57-79) genes.

FIG. 5A-C shows an alignment of Ag43 nucleotide sequences (SEQ ID NOs:80-112). The Ag43 sequences shown here encode, in the +3 reading frame, amino acids corresponding to amino acids 718-822 of SEQ ID NO:81.

### DETAILED DESCRIPTION OF EMBODIMENTS

Our initial studies involve culture-independent surveys of microbial diversity. Our survey forms a prelude to targeted cultivation of bacteria, leading to our finding that SPATE and Ag43 are useful microbial markers of IBD. However, although our invention arose from our study and finds basis in our scientific results, the invention should not be limited to the specific results. This is understood to those working in the field of microbiology as it pertains to inflammatory bowel disease.

We note that, where microorganisms are identified using culture-independent methods as being associated with disease, there may be advantages to culturing them so that their virulence mechanisms can be evaluated.

The work described in the example here involve the use of ribosomal intergenic spacer analysis (RISA, ref 13) of biopsy tissue to identify bands that were consistently associated with IBD tissue. We used highly focused cultivation methods, including resuscitation methods, to specifically culture Enterobacteriaceae.

### (I) Serine Protease Autotransporter (SPATE) and Antigen 43 (AG43)

The term “SPATE” in the present context means nucleic acids and polypeptides which are members of the serine protease autotransporter family and which are homologs of the nucleotide sequence identified as SEQ ID NO:1 and the amino acid sequence identified as SEQ ID NO:2. The term “Ag43” in the present context means nucleic acids and polypeptides which are members of the autotransporter family and which are homologs of the nucleotide sequence identified as SEQ ID NO:80 and the amino acid sequence identified as SEQ ID NO:81. Autotransporter proteins including the SPATE and Ag43 family are described in ref 28.

Homologs of the SPATE family include, for example, the SPATE sequences identified in the Genbank accession numbers: AF056581, AF218073, AF297061, AJ278144, AJ586888, AX276281, AY163491, AY258503, AY604009, DD002707, U69128, X97542, Y13614, NZ_AAJV01000028, Sat_AX702523. The homolog sequences also include those shown as SEQ ID NOS:1, 2 and 57-71.

We contemplate “SPATE” in the present invention to encompass naturally occurring homologs and sequence variants of SEQ ID NO:1 and 2. A SPATE homolog possesses the three domains that are typical of SPATE autotransporters: an unusually long signal sequence of about 49 amino acids, a passenger domain containing a consensus serine protease active site (GDSGSP or GDSGSG); and a C-terminal autotransporter domain. “Homologous amino acid sequence” or “variant amino acid sequence” is any polypeptide which is encoded, in whole or in part, by a nucleic acid sequence which hybridizes at 25-35° C. below critical melting temperature (Tm), to any portion of the nucleic acid sequence of SEQ ID No: 1. A homologous amino acid sequence is one that differs from an amino acid sequence shown in SEQ ID No: 2 by one or more conservative amino acid substitutions. Such a sequence encompasses those variants which retain at least one inherent characteristics of the polypeptide such as immunogenicity, serine protease activity, haemaglutinin activity, mucinase activity, elastase activity, cytotoxic effects on cells, elastase activity, lipoprotein cleavage activity, coagulation factor V cleavage activity, the ability to degrade the barrier function of the gut, and the ability to cleave proteins in the enterocyte. Such a sequence is contemplated as being at least 75%, 80%, 90% or 95% identical to SEQ ID No: 2. We contemplate a homolog or variant sequence to differ from the sequence of reference by a majority of conservative amino acid substitutions, i.e. substitutions among amino acids of the same class.

We contemplate “Ag43” in the present invention to encompass naturally occurring homologs and sequence variants of SEQ ID NO:80 and 81. “Homologous amino acid sequence” or “variant amino acid sequence” is any polypeptide which is encoded, in whole or in part, by a nucleic acid sequence which hybridizes at 25-35° C. below critical melting temperature (Tm), to any portion of the nucleic acid sequence of SEQ ID No: 80. A homologous amino acid sequence is one that differs from an amino acid sequence shown in SEQ ID No: 81 by one or more conservative amino acid substitutions. Such a sequence encompasses those variants which retain at least one inherent characteristics of the polypeptide such as immunogenicity, auto-aggregation activity, cell to cell aggregation, the ability to induce a frizzy colony morphology and the ability to form a biofilm (see refs 27 and 39). Such a sequence is contemplated as being at least 75%, 80%, 90% or 95% identical to SEQ ID No: 81. We contemplate a homolog or variant sequence to differ from the sequence of reference by a majority of conservative amino acid substitutions, i.e. substitutions among amino acids of the same class.

Homologs of the Ag43 family include, for example, the Ag43 sequences identified in the Genbank accession numbers: AE005174, AE014075, AF233271, AF233272, AJ303141, AJ586887, AJ586888, AJ617685, AP009048, AR580480, AX370193, AX702425, AX702524, AY857617, BA000007, BD184766, BD195283, BD444174, CP000243, CS148067, U00096, U24429, X16664. The homolog sequences also include those shown as SEQ ID NOS: 80-104.

Homology is measured using sequence analysis software such as Sequence Analysis Software Package of the Genetics Computer Group, University of Wisconsin Biotechnology Center, 1710 University Avenue, Madison, Wis. 53705. Amino acid sequences are aligned to maximize identity. Gaps may be artificially introduced into the sequence to attain proper alignment.

In the present context, stringent conditions are achieved for both pre-hybridizing and hybridizing incubations (i) within 4-16 hours at 42° C., in 6 ×SSC containing 50% formamide, or (ii) within 4-16 hours at 65° C. in an aqueous 6×SSC solution (1 M NaCl, 0.1 M sodium citrate (pH 7.0)). Typically, hybridization experiments are performed at a temperature from 60 to 68° C., e.g. 65° C. At such a temperature, stringent hybridization conditions can be achieved in 6×SSC, preferably in 2×SSC or 1×SSC, more preferably in 0.5×SSc, 0.3×SSC or 0.1×SSC (in the absence of formamide). 1×SSC contains 0.15 M NaCl and 0.015 M sodium citrate.

In particular, we contemplate SPATE sequences which contain the conserved serine protease motif GDSGSP or GDSGSG (corresponding to amino acids 195-200 of SEQ ID NO:2).

In the context of diagnosis or prognosis or evaluating the effectiveness of a treatment for IBD, i.e. in the context where SPATE or Ag43 is to be detected in a sample from a subject, we contemplate the naturally occurring SPATE or Ag43 sequences, examples for which are set out above.

In the context of testing potential therapeutic agents for treating IBD, we contemplate using not only SPATE or Ag43 polypeptides and nucleic acids having naturally-occurring sequences, but also SPATE or Ag43 fragments; in particular fragments containing the conserved serine protease motif GDSGSP or GDSGSG.

The term “isolated polynucleotide or nucleic acid or polypeptide” is defined as a polynucleotide or nucleic acid or polypeptide removed from the environment in which it naturally occurs. For example, a naturally-occurring DNA molecule present in the genome of a living bacteria or as part of a gene bank is not isolated, but the same molecule separated from the remaining part of the bacterial genome, as a result of, e.g., a cloning event (amplification), is isolated. Typically, an isolated DNA molecule is free from DNA regions (e.g., coding regions) with which it is immediately contiguous at the 5′ or 3′ end, in the naturally occurring genome. Such isolated polynucleotides may be part of a vector or a composition and still be defined as isolated in that such a vector or composition is not part of the natural environment of such polynucleotide.

The polynucleotide for use in certain aspects of the invention is either RNA or DNA (cDNA, genomic DNA, or synthetic DNA), or modifications, variants, homologs or fragments thereof. The DNA is either double-stranded or single-stranded, and, if single-stranded, is either the coding strand or the non-coding (anti-sense) strand. By “polypeptide” or “protein” is meant any chain of amino acids, regardless of length or post-translational modification (e.g., glycosylation or phosphorylation). Both terms are used interchangeably in the present application. Polynucleotide and nucleic acid are interchangeable terms as used herein.

Partial sequences of SPATE or Ag43, e.g. of SEQ ID No:2 or 81, or their homologous amino acid sequences, are inherent to the full-length sequences. Such polypeptide fragments preferably are at least 12 amino acids in length, preferably at least 15, 20, 25, 30, 35, 40, 45, 50 amino acids, more preferably at least 55, 60, 65, 70, 75 amino acids, and most preferably at least 80, 85, 90, 95, 100 amino acids in length.

In the present context, fusion polypeptides may be useful, for example for testing potential therapeutic agents that may bind or inhibit SPATE or Ag43. A fusion polypeptide is one that contains a polypeptide or a polypeptide derivative of the invention fused at the N- or C-terminal end to any other polypeptide (hereinafter referred to as a peptide tail). A simple way to obtain such a fusion polypeptide is by translation of an in-frame fusion of the polynucleotide sequences, i.e., a hybrid gene.

### (II) Assays for SPATE and Ag43 Nucleic Acids

The assay for SPATE and Ag43 can involve direct assay of nucleic acid levels, such as mRNA levels to measure gene expression, or involve direct assay of DNA, for example by PCR, to gauge the number of gene copies, thereby estimating the level of SPATE- or Ag43-containing, and possibly pathogenic, microbes.

The SPATE or Ag43 nucleic acid molecules are also useful as hybridization probes for determining the presence, level, form and distribution of SPATE or Ag43 nucleic acid and SPATE- or Ag43-containing microbes. Experimental data as provided herein indicates that SPATE or Ag43 and virulent enterobacteria expressing SPATE or Ag43 are associated with IBD. Accordingly, probes based on SPATE or Ag43 sequences can be used to detect the presence of, or to determine levels of, SPATE or Ag43 in cells, tissues, and in organisms of the gut. The nucleic acid whose level is being determined can be DNA or RNA. Accordingly, probes corresponding to the peptides described herein can be used to assess expression and/or gene copy number in a given cell, tissue, or organism. These uses are relevant for diagnosis of disorders involving an increase or decrease in SPATE or Ag43 expression, or involving an increase or decrease in SPATE or Ag43 DNA-containing microbes, relative to normal results.

Nucleic acids can be detected by methods known in the art. RNA may be detected by for example Northern analysis or by the reverse transcriptase-polymerase chain reaction (RT-PCR) method (see for example Sambrook et al (1989) Molecular Cloning: A Laboratory Manual (second edition), Cold Spring Harbor Laboratory Press, Cold Spring Harbor, N.Y., USA). DNA is routinely detectable by PCR, Southern hybridizations and in situ hybridization.

Probes can be used in a diagnostic test for identifying samples containing SPATE or Ag43 or SPATE- or Ag43-expressing microbes, such as by measuring a level of nucleic acid encoding SPATE or Ag43 in a sample of microbes from a subject.

Detection of SPATE or Ag43 nucleic acids may be designed such that specific sub-families of SPATE or Ag43 are detected, e.g. those of enteric bacteria, enteric E. coli, or virulent enteric strains of E. coli. It is also useful to detect SPATE or Ag43 nucleic acids specific to certain sub-families, e.g. SPATE or Ag43 specific to enteric bacteria, enteric E. coli, virulent enteric strains of E. coli, or E. coli of the B2 or D genotype or the B2+D genotype. By choosing the regions of SPATE or Ag43 sequences which are conserved or unique to the various sub-families, one can target detection of certain SPATEs or Ag43s. For example, a probe or PCR primers complementary to a SPATE or Ag43 sequence that is conserved among enteric E. coli can be used to detect the presence of SPATE or Ag43, and thereby the presence of enteric E. coli, in the sample, which is diagnostic or prognostic of IBD.

### (III) Assays for SPATE OR AG43 Polypeptide

SPATE or Ag43 proteins are useful targets for diagnosing IBD or predisposition to IBD. The invention provides methods for detecting the presence, or levels of, the protein (or encoding mRNA) in a sample of cell, tissue or body fluid containing enteric bacteria. Experimental data as provided herein shows SPATE or Ag43 to be a microbial indicator of IBD. Thus to diagnosis or prognosis of IBD involves contacting an enteric microbe-containing sample with a compound capable of interacting with SPATE or Ag43, such that the interaction can be detected. Such an assay can be provided in a single detection format or a multi-detection format such as an antibody chip array.

SPATE protein levels may be detected either directly using affinity reagents, e.g. an antibody or fragment thereof (for methods, see for example Harlow, E. and Lane, D (1988) Antibodies: A Laboratory Manual, Cold Spring Harbor Laboratory Press, Cold Spring Harbor, N.Y.)) or by assaying the protein's activity such as serine protease activity (ref 38), haemaglutinin activity, mucinase activity, elastase activity (ref 29 & 30), cytotoxicity (ref 31-33), elastase activity (ref 27-30), lipoprotein cleavage activity (ref 27-30), coagulation factor V cleavage activity.

Ag43 protein levels may be detected either directly using affinity reagents, e.g. an antibody or fragment thereof (for methods, see for example Harlow, E. and Lane, above) or by assaying the protein's activity such as auto-aggregation activity, cell to cell aggregation, the ability to induce a frizzy colony morphology and the ability to form a biofilm (see refs 27 and 39).

One useful agent for detecting SPATE or Ag43 protein in a sample is an antibody capable of selectively binding to SPATE or Ag43. Binding may be selective with respect to specific sub-families of SPATE or Ag43, e.g. those of enteric bacteria, enteric E. coli, or virulent enteric strains of E. coli, or E. coli of the B2 or D or the B2+D genotype. By choosing antibodies immunospecific for the regions of SPATE or Ag43 which are conserved or unique to the various sub-families, one can target detection of certain SPATEs or Ag43s. For example, an antibody immunospecific for a SPATE or Ag43 epitope that is a conserved sequence among enteric E. coli can be used to detect enteric E. coli in the sample, which is diagnostic or prognostic of IBD.

For detecting SPATE or Ag43 protein and thereby diagnosing or prognosing IBD in a subject, an antibody may be use in a method that includes contacting an enteric bacteria-containing sample from a subject with an anti-SPATE or anti-Ag43 antibody, under conditions suitable to form a complex between SPATE or Ag43 and the antibody; and detecting the presence or absence of the complex. The presence of the complex indicates that the subject has or is susceptible to developing IBD. The presence or absence of the complex can be detected, for example, with a detectable secondary antibody that has specificity for a class determining portion of the primary antibody. The term “complex” is used synonymously here with “immune complex” and means an aggregate of two or more molecules that results from specific binding between an antigen (SPATE or Ag43) and an antibody.

An antibody of the invention is either polyclonal or monoclonal. Monospecific antibodies may be recombinant, e.g., chimeric (e.g., constituted by a variable region of murine origin associated with a human constant region), humanized (a human immunoglobulin constant backbone together with hypervariable region of animal, e.g., murine, origin), and/or single chain. Both polyclonal and monospecific antibodies may also be in the form of immunoglobulin fragments, e.g., F(ab)′2 or Fab fragments. The antibodies of the invention are of any isotype, e.g., IgG or IgA, and polyclonal antibodies are of a single isotype or a mixture of isotypes.

Antibodies against the polypeptides, homologs or fragments of the present invention are generated by immunization of a mammal with a composition comprising said polypeptide, homolog or fragment. Such antibodies may be polyclonal or monoclonal. Methods to produce polyclonal or monoclonal antibodies are well known in the art. For a review, see “Antibodies, A Laboratory Manual, Cold Spring Harbor Laboratory, Eds. E. Harlow and D. Lane (1988), and D. E. Yelton et al., 1981. Ann. Rev. Biochem. 50:657-680. For monoclonal antibodies, see Kohler & Milstein (1975) Nature 256:495-497.

The antibodies of the invention, which are raised to a polypeptide or polypeptide derivative of the invention, are produced and identified using standard immunological assays, e.g., Western blot analysis, dot blot assay, or ELISA (see, e.g., Coligan et al., Current Protocols in Immunology (1994) John Wiley & Sons, Inc., New York, N.Y.). The antibodies are used in diagnostic methods to detect the presence of a SPATE or At43 antigen in a sample, such as a biological sample. The antibodies are also used in affinity chromatography for purifying a polypeptide or polypeptide derivative of the invention.

Those skilled in the art will readily understand that the immune complex is formed between a component of the sample and the antibody, polypeptide, or polypeptide derivative, whichever is used, and that any unbound material is removed prior to detecting the complex. It is understood that a polypeptide reagent is useful for detecting the presence of anti-SPATE or anti-Ag43 antibodies in a sample, while an anti-SPATE or anti-Ag43 antibody is useful for screening a sample, such as a gastric extract or biopsy, for the presence of SPATE or Ag43 polypeptides.

A secondary antibody can be, for example, an anti-IgA secondary antibody, an anti-IgG secondary antibody, or a combination of anti-IgA and anti-IgG secondary antibodies.

In vitro techniques for detection of SPATE or Ag43 include enzyme linked immunosorbent assays (ELISAs), Western blots, immuno-precipitations and immunofluorescence using a detection reagent, such as an antibody or protein binding agent. Alternatively, the protein can be detected in vivo in a subject by introducing into the subject a labeled antibody or other types of detection agent. For example, the antibody can be labeled with a radioactive marker whose presence and location in a subject can be detected by standard imaging techniques. Particularly useful are methods that detect the allelic variant of a peptide expressed in a subject and methods which detect fragments of a peptide in a sample.

As used herein, an antibody selectively binds a target peptide when it binds the target peptide and does not significantly bind to unrelated proteins. An antibody is still considered to selectively bind a peptide even if it also binds to other proteins that are not substantially homologous with the target peptide so long as such proteins share homology with a fragment or domain of the peptide target such that there are shared epitopes. In this case, it would be understood that antibody binding to the peptide is still selective despite some degree of cross-reactivity.

As used herein, an antibody is defined in terms consistent with that recognized within the art: they are multi-subunit proteins produced by a mammalian organism in response to an antigen challenge. The term “antibody” as used herein include polyclonal antibodies and monoclonal antibodies, as well as fragments of such antibodies, including, but not limited to, Fab or F(ab′)₂, and Fv fragments.

Detection of an antibody of the present invention, and thus detection of an antibody-SPATE or antibody-Ag43 complex, can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³I, ³⁵S or ³H.

The antibodies can be used to isolate SPATE or Ag43 or fragments thereof by standard techniques, such as affinity chromatography or immunoprecipitation. The antibodies can facilitate the purification of the protein from natural sources and recombinantly produced protein expressed in host cells. In addition, such antibodies are useful to detect the presence of one of the proteins of the present invention in cells or tissues to determine the pattern of expression of the protein among various tissues in an organism and over the course of normal development. Such antibodies can also determine the pattern of colonisation of SPATE-expressing microbes or Ag43-expressing microbes along the gastro-intestinal tract.

Experimental data as provided herein indicates that SPATE or Ag43 and virulent enterobacteria expressing SPATE or Ag43 are associated with IBD. Antibodies to SPATE or Ag43 can be used to detect SPATE or Ag43 protein in situ, in vitro, or in a lysate or supernatant of an enteric bacteria-containing sample in order to evaluate the abundance and pattern of expression. The antibodies can also be used to assess a predisposition toward IBD, i.e. a prognosis of IBD.

### (IV) SPATE and IBD; AG43 and IBD

SPATEs are potentially important in IBD because they exhibit functions like degradation of the barrier function of the gut, and cleavage of proteins in the enterocyte, all of which are phenotypes associated with IBD (ref 28). For example Vat, Pic, and Pic-like have haemaglutinin, mucinase, and elastase activity (ref 29, 30), Sat has cytotoxic effects (ref 31-33) on cells as well as elastase activity (ref 27-30), and EspP cleaves lipoproteins (ref 27-30). Functional studies on SPATE have demonstrated that Pic and Sat can cleave coagulation factor V, potentially linking it to haemorrhagic events in the gut. Additionally, Pic is thought be involved in colonisation of E. coli to intestinal tissue (ref 34). Ag43 is a surface adhesin that promotes bacterial biofilm formation due to cell-to-cell aggregation (ref 27).

SPATE or Ag43 can thus be used as a diagnostic marker for IBD, or to determine susceptibility to developing IBD in a subject. The presence of SPATE or Ag43 in an enteric bacteria-containing sample from a subject compared to a control indicates that the subject has IBD or is susceptible to developing IBD.

As used herein, the term “subject” means any animal capable of having inflammatory bowel disease, including a human, non-human primate, rabbit, rat or mouse, especially a human. A subject can have one or more symptoms of Crohn's disease or ulcerative colitis, or may be asymptomatic. The term “subject having IBD” means a subject having the clinical features of IBD as defined herein. The term “susceptible to IBD” as indicated by the presence of SPATE or Ag43 in the gut micro flora of the subject means a reduced ability to resist IBD-causing factors, as compared with an individual from whom a sample is obtained that does not contain a significant level of SPATE or Ag43 or SPATE- or Ag43-expressing microbes. Susceptibility to IBD in a subject does not mean the subject will develop IBD, but that the subject has an increased probability of having symptoms of IBD in the future.

Inflammatory bowel disease (IBD) encompasses a group of diseases such as ulcerative colitis (UC) and Crohn's disease (CD). IBDs can be difficult to diagnose. An initial diagnosis, made on the basis of medical history and physical examination, is generally confirmed via imaging tests to look at the intestines and laboratory culture tests to rule out bacterial, viral and parasitic infections. Crohn's disease affects some areas of the intestines and not others. Ulcerative colitis is more dispersed. Endoscopy is used to take a biopsy of intestinal tissue, which can be used to identify the deep inflammation of the bowel that is characteristic of Crohn's disease. X-rays (after oral or rectal ingestion of Barium), computed tomography (CT) scan, and magnetic resonance imaging (MRI) may be helpful in locating fistulas. A stool analysis (including a test for blood in the stool) is often performed, depending on symptoms, to look for blood and signs of bacterial infection. Blood and urine tests may be done to check for anemia, high white cell counts, or malnutrition; all these are signs of IBDs.

Murine models of inflammatory bowel disease can be used in certain aspects of the invention, e.g. to evaluate a potential treatment for IBD using a compound identified by screening with SPATE or Ag43. Animal models are known to those in the art, for example, mice with targeted disruption of the gene encoding the alpha subunit of the G-protein Gi2 exhibit features of human bowel disease. Mice deficient in IL-10 and mice deficient in IL-2 also have colitis-like disease.

A suitable sample is any sample containing enteric bacteria. Sources for samples include gut tissue biopsy, intestinal mucosa, stool or fecal matter, or intestinal wash. In particular, the sample may be a colonoscopy tissue biopsy from the lower gastrointestinal (GI) tract. Biopsies resected from the gastro-intestinal tract and from an area believed to be exhibiting signs of the disease may also be useful as the sample source. The sample may be used directly in the methods described herein. Alternatively, the sample may be processed e.g. to remove particulate matter or to remove mucus etc. as appropriate for a chosen technique.

In the methods described herein, one may determine the level of SPATE or Ag43 in the sample, rather than simply detecting the presence of SPATE or Ag43 compared to the absence of SPATE or Ag43. A higher level of SPATE or Ag43 compared to a cut-off value would indicate that the subject has IBD or or is susceptible to developing IBD. By “higher level”, we mean a quantitative rather than qualitative difference since “absence” or “presence” are relative terms, a test sample result being always to be compared with a control. An appropriate control are samples from subjects free of IBD symptoms, or possibly samples from the same IBD subject but obtained from a region of the GI tract that contains enteric bacteria but is free of inflammation or any sign of IBD. To normalize the SPATE or Ag43 values obtained from the assays, one may assay for a protein or gene which is known to be present equivalently in both IBD and non-IBD subjects. The SPATE or Ag43 values may be normalized against such control values. Since SPATE or Ag43 may be present in non-pathogenic bacteria, there may be a baseline level of SPATE or Ag43 in non-IBD subjects. Such a baseline level establishes a cut-off value for determining whether a subject has more-than-normal SPATE or Ag43.

The invention also encompasses commercial packages or kits for diagnosing IBD or determining susceptibility to developing IBD in a subject. The package comprises an agent for detecting SPATE or Ag43 in an enteric bacteria-containing sample from the subject. The package may also contain instructions for using the agent to detect SPATE or Ag43 in the sample, thereby diagnosing or prognosing IBD in the subject. If SPATE or Ag43 nucleic acid is to be detected, the agent may be a nucleic acid probe or a set of primers for use in PCR amplification. If SPATE or Ag43 protein is to be detected, the agent may be an antibody immunospecific for SPATE or Ag43; the package may also contain secondary antibodies to detect the SPATE-antibody or Ag43-antibody complex. The package may also contain SPATE or Ag43 DNA or protein or cells expressing SPATE or Ag43, in unit amounts suitable as standards against which the test results are assessed. The package may also contain reagents or materials for detecting a normalizing protein or gene (one which is known to be present equivalently in both IBD and non-IBD subjects) against which the SPATE or Ag43 test results are assessed.

### (V) Screening Assays for Potential Therapy

Nucleic acid expression assays are useful for drug screening to identify compounds that modulate SPATE or Ag43 gene expression or modulate growth of SPATE- or Ag43-expressing microbes. The invention thus provides a method for identifying a compound that can be used to treat a disorder of the gastro-intestinal tract associated with expression of SPATE or Ag43, particularly biological and pathological processes that involve SPATE or Ag43. The method typically includes assaying the ability of the compound to modulate expression of the pertinent SPATE or Ag43 gene and thus identify a compound that can be used to treat a disorder characterized by SPATE or Ag43 gene expression.

Thus SPATE or Ag43 may be used as a target in screening assays to identify compounds that are useful as inhibitors of SPATE or Ag43 for the prevention or treatment of IBD. In some embodiments, such an assay may comprise the steps of: (a) providing a test compound; (b) providing a source of SPATE or Ag43; and (c) measuring SPATE or Ag43 activity in the presence versus the absence of the test compound. A lower measured activity in the presence of the test compound would indicate that the compound is an inhibitor of SPATE- or Ag43-dependent activity and may be useful for the prevention and/or treatment of IBD.

“SPATE activity” as used herein refers to any type of observed phenomenon which can be attributed to SPATE. Such activity includes serine protease activity (ref 38), haemaglutinin activity, mucinase activity, elastase activity (ref 29 & 30), cytotoxicity (ref 31-33), elastase activity (ref 27-30), lipoprotein cleavage activity (ref 27-30), coagulation factor V cleavage activity. Another inherent activity that can be assayed for is immunogenicity. A test compound that can mitigate or block a SPATE-specific epitope may be effective. “Ag43 activity” as used herein refers to any type of observed phenomenon which can be attributed to Ag43. Such activity includes auto-aggregation activity, cell to cell aggregation, the ability to induce a frizzy colony morphology and the ability to form a biofilm (see refs 27 and 39). Another inherent activity that can be assayed for is immunogenicity. A test compound that can mitigate or block a Ag43-specific epitope may be effective.

The assay may be carried out in vitro utilizing a source of SPATE or Ag43 which may comprise naturally isolated or recombinantly produced SPATE or Ag43, in preparations ranging from crude to pure. Such assays may be performed in an array format.

The assay may in an embodiment be performed using an appropriate host cell as a source of SPATE or Ag43. Such a host cell may be prepared by the introduction of DNA encoding SPATE or Ag43 into the host cell and providing conditions for the expression of SPATE or Ag43.

SPATE or Ag43 and fragments, particularly those SPATE fragments comprising the conserved protease motif GDSGSP or GDSGSG, are also useful in drug screening assays, in cell-based or cell-free systems. Cell-based systems can be native, i.e. microbes that normally express SPATE or Ag43. In an alternate embodiment, cell-based assays involve recombinant host cells expressing SPATE or Ag43 or their fragments.

SPATE or Ag43 can be used to identify compounds that modulate its role in IBD. SPATE or Ag43 and appropriate variants and fragments can be used in high-throughput screens to assay candidate compounds for the ability to bind to SPATE or Ag43 and their fragments. These compounds can be further screened against a functional SPATE or Ag43 to determine the effect of the compound on SPATE or Ag43 activity. Further, these compounds can be tested in animal systems to determine activity/effectiveness.

Binding and/or activating compounds can also be screened by using fusion proteins in which the amino terminal domain, or parts thereof, and the carboxy terminal domain, or parts thereof, can be replaced by heterologous polypeptides. These are generally referred to as chimeric or fusion proteins.

SPATE or Ag43 and fragments, particularly those SPATE fragments comprising the conserved protease motif GDSGSP or GDSGSG, are also useful in competition binding assays in methods designed to discover compounds that interact with SPATE or Ag43 (e.g. binding partners and/or ligands). Thus, a compound is exposed to SPATE or Ag43 and fragments under conditions that allow the compound to bind or to otherwise interact with the polypeptide. A known binding partner such as a monoclonal antibody to SPATE or to Ag43 is also added to the mixture. If the test compound interacts with SPATE or Ag43, it may decrease the amount of complex formed between SPATE or Ag43 and the known binding partner. This type of assay is particularly useful in cases in which compounds are sought that interact with specific regions of SPATE or Ag43. Thus, the binding partner that competes with the test compound is designed to bind to peptide sequences corresponding to the region of interest in SPATE or Ag43.

The above-described assay methods may further comprise determining whether any compound so identified can be used for the prevention or treatment of IBD, such as examining their effect(s) on disease symptoms in suitable animal model systems. Furthermore, one can examine their effect(s) on the SPATE- or Ag43-expressing microbe to determine whether the compound adversely affects pathogenic microbes, e.g. inhibiting their growth and functioning as a microbe-specific antibiotic.

### (VI) Cultivating Enteric Bacteria and Phylogenetic Identification of IBD-Associated Microbes

We found that we could culture bacteria from untreated biopsy tissue by resuscitating the sample in buffered peptone water (a media used for the preliminary, non-selective enrichment of bacteria, particularly pathogenic Enterobacteriaceae, from foodstuffs and other materials; available for example at Genpharm Inc. 85 Advance Road, Etobicoke, Ontario M8Z 2S9 Canada).

We further worked on a culture-independent strategy for picking up bacteria that are present preferentially on IBD tissue. We applied nucleic acid-based techniques to identify DNA segments more commonly present in IBD than in controls. Our method involves: (i) amplifying DNA from an enteric bacteria-containing sample from a subject having IBD; (ii) amplifying DNA from an enteric bacteria-containing sample from a subject free of IBD; (iii) comparing the amplified DNA obtained from step (i) with amplified DNA obtained from step (ii) to mark out DNA that is associated with the subject having IBD; and (iv) performing phylogenetic analysis to identify bacteria containing the DNA associated with the subject having IBD.

The overall goal of our strategy is to obtain as diverse a survey of the micro flora as possible, and identify from the results those associated with IBD. To obtain a diverse cross section of the micro flora, the method involves obtaining microbial nucleic acids in regions that are variable, in order that the polymorphisms can point to the phylogeny of the microbe. One way to do this involves targeting a region flanked by conserved sequences of enteric bacteria. The polymorphic analysis can be performed directly on the sample, or preferably on extracted DNA. Suitable DNA extraction techniques for extracting the total DNA from the various type of samples are well known, and the appropriate method would easily be determined.

The DNA can be amplified by known methods, such as for example, the PCR method. In PCR, preferably a primer would be directed towards a conserved region to ensure that the largest population of micro flora DNA is amplified, while the area amplified includes a less conserved region, thereby allowing a broad polymorphic analysis. Suitable examples might, for example, include the 16S rRNA gene, 23S rRNA gene or the region between the 16S and 23S rRNA genes. Any form of polymorphic analysis is suitable. The more variable products that are detectable, the more determinate the analysis will be. For example, a restriction fragment length polymorphism analysis could be performed over the variable region of the 16S rRNA gene.

Comparison of the polymorphic analysis of the DNA from the sample of unknown origin to the DNA of the sample of known origin can be achieved using a variety of methods. For example, for a restriction fragment length polymorphism analysis, comparison may be achieved by a visual comparison of an autoradiograph of a polyacrylamide gel electrophoresis, or alternatively the PCR product could be fluorescently-tagged and then laser detected and the electropherogram may be visually compared. Alternatively, the polymorphic profiles may be compared mathematically. A suitable method for determining E. coli phylogenetic group is described in ref 15.

### (VII) Experimental Basis

### Materials and Methods

Study subjects: We utilized 84 biopsies from 15 controls, 13 Crohn's disease (CD) patients (3 with ileal disease, 6 with ileocolonic disease and 4 with isolated colonic disease), and 19 ulcerative colitis (UC) patients (3 with proctitis, 8 with left sided colitis, and 8 with pancolitis) from a population-based case-control study undertaken at the University of Manitoba (table 1) as described (ref 14).

In brief, a population-based study refers to a process by which selection of study subjects proceeds by accounting for bases related to various factors like lifestyle (e.g. smoking), geographic location (e.g. urban vs rural), age, gender, or ethnicity. In IBD research it is challenging to obtain untainted biopsy controls because endoscopy is normally only performed on persons when it is clinically required. The controls were true controls in the sense that the subjects voluntarily submitted to endoscopy and were drawn from the same population-based study. No antibiotics were provided to any of the subjects in the six weeks prior to the colonoscopy.

TABLE 1
Biopsy samples used in this study*

| | Controls | | CD | | UC | | IBD | |
|---|---|---|---|---|---|---|---|---|
| | (15 subjects | | (13 patients | | (19 patients | | (32 patients | |
| | 28 tissues) | | 27 tissues) | | 29 tissues) | | 56 tissues) | |
| Site | End. | Hist. | End. | Hist. | End. | Hist. | End. | Hist. |
| Rectum | 0 (13) | 0 (13) | 5 (13) | 5 (13) | 9 (10) | 9 (10) | 14 (23) | 14 (23) |
| Caecum | 0 (15) | 0 (15) | 4 (9) | 6 (9) | 2 (15) | 4 (15) | 6 (24) | 10 (24) |
| Colon | 0 (0) | 0 (0) | 3 (5) | 3 (5) | 2 (4) | 2 (4) | 5 (9) | 5 (9) |

*Endoscopic (End.) and histological (Hist.) examination was made on biopsies and only histologically positive samples were considered inflamed. Total numbers of biopsies for each group are presented in parentheses, and the numbers not in parentheses represent inflamed biopsies.

Colonoscopy plus biopsies: Following a standard oral Fleet® Phospho-soda® treatment, biopsies were taken from the caecum the rectum. In subjects with a previous caecal resection, biopsies were obtained from the right colon distal to the ileocolonic anastmosis. All biopsies were snap frozen in liquid nitrogen and stored at −70° C. Biopsies were subject to standard histological staining with haematoxylin and eosin for evaluation of inflammation. A site was considered inflamed if it had histological evidence of inflammation and was considered uninflamed if it was histologically normal.
DNA extraction for RISA analysis: Tissue samples were suspended in 150 μl lysis buffer [10 mM Tris-HCl, pH 8.0; 5 mM EDTA, pH 8.0; 4 M guanidinium isothiocyanate (GITC), pH 7.5; 50 g Sarcosyl/L, 2.5 g SDS/L, 5 g sodium citrate/L and 5 g Triton X-100/L]. 300 μl of chloroform and Tris-saturated phenol (pH 6.9) were added to each tube. The samples were placed at −20° C. for 1 h. Subsequently, samples were centrifuged in microfuge tubes at 4° C. for 20 min at 10,000×g. Supernatants were transferred to fresh tubes. Isopropanol to ¼ volume of the supernatants was added and the mixtures loaded onto silica-cellulose membranes in columns. Samples were allowed to filter through the membrane by gravity. The membranes were washed twice with 300 μl 95% ethanol (by gravity). DNA was eluted with 400 μl hot (about 75° C.) TE buffer (by gravity) and precipitated with two portions of 95% ethanol. The resulting pellets were suspended in 25 μl 0.5× TE buffer (pH 8.0) and stored at −20° C. until further analysis.

Primer sequences used for RISA are listed in table 2 and amplified intergenic transcribed spacers between the 16S and 23S rDNA13. PCR products were subjected to electrophoresis using 2% agarose. DNA fragments only found in UC and CD were purified from agarose gels, cloned into the pCR®2.1-TOPO TA vector (Invitrogen) and sequenced. Standard bioinformatics analysis was used to taxonomically classify the sequence fragment.

Bacterial cultures: Once RISA analysis had determined that the bacteria that appeared in UC and CD and not in controls were E. coli, targeted bacterial cultivation was carried out. All cultivation of the bacteria was with untreated biopsy tissue and no procedures were used to wash the tissues or to remove mucus. To ensure as many E. coli cells as possible were cultured, resuscitation in buffered peptone water was performed, followed by decimal dilution and culturing on chromogenic E. coli/coliform medium (Oxoid CM0956). Resuscitation was by incubation of the biopsy in 1 ml of 100 mM buffered peptone water for 16 hours at 37° C. Ten μl droplets were pipetted (Maxipettor, Eppendorf) onto media, allowed to dry, and then inverted and incubated at 37° C. After 18 hours, E. coli (purple colonies) and non-E. coli coliforms (blue, pink, and white colonies) were counted separately.

Colour differentiation on chromogenic agar is a good first approximation of E. coli identity. Five putative E. coli (purple colonies) were picked from each positive tissue and sub-cultured in LB broth, then recultured on E. coli/coliform medium, and tested for reactivity to indole, methyl red, Vogues Proskauer, and citrate utilisation to differentiate E. coli from non-E. coli. The 16S rDNA gene sequences for all the non-E. coli were determined using standard primers (table 2).

DNA extraction from bacterial cultures: For DNA extraction from cultures, 1 ml suspensions of each culture were centrifuged and pellets were suspended in lysis buffer and subsequently mixed with chloroform and Tris-saturated phenol. All other steps were the same as for the extraction of DNA from tissue samples.

Molecular analysis of bacterial cultures: All primer sequences used for molecular analysis of bacterial cultures are listed in table 2. Primers were either modified from published primers, or newly designed for the purposes of our studies. PCR was by standard methods after optimisation for specificity of primers pairs by adjusting annealing temperature and salt concentration. Amplified products were run on agarose gels as described above.

TABLE 2
Primers used

| | | |
|---|---|---|
| Primer sequences | Target sequences | SEQ ID NO |

| | | |
|---|---|---|
| Autotransporters | | |
| SPATE1 5′ GAGGTCAACAACCTGAACAAACGTATGGG | The genes encoding serine | 3 |
| SPATE2 5′ CCGGCACGGGCTGTCACTTTCCAG | protease autotransporters | 4 |
| | (SPATE) | |
| E. coli phylogenetic groups | | |
| ChuAf 5′ CGGACGAACCAACGGTCAGGAT | The chuA gene is required for | 5 |
| ChuAr 5′ TGCCGCCAGTACCAAAGACACG | heme transport in E. coli | 6 |
| | O157:H7 | |
| Yjaf 5′ CGTGAAGTGTCAGGAGACGCTGC | The yjaA gene coding for | 7 |
| Yjar 5′ TGCGTTCCTCAACCTGTGACAAACC | protein of unknown function | 8 |
| Tsp1 5′ GGGAGTAATGTCGGGGCATTCAG | Tsp encodes for a putative | 9 |
| Tsp2 5′ CATCGCGCCAACAAAGTATTACGCAG | DNA fragment (TSPE4.C2) in E. | 10 |
| | coli | |
| E. coli toxins | | |
| Cnff 5′ AGTACTGACACTCACTCAAGCCGC | Cytotoxic necrotising factors | 11 |
| Cnfr 5′ GCAGAACGACGTTCTTCATAAGTATCACC | (Cnf1 and Cnf2) | 12 |
| IpgDf 5′ CGACTTCTCTTCTGACGCCGAC | ipgD gene modulates entry of | 13 |
| IpgDr 5′ CAACATTCCTCCAGCCTAAGCCC | bacteria into epithelial | 14 |
| | cells | |
| Vt1f 5′ CGCATAGTGGAACCTCACTGACGC | Verocytotoxin 1 | 15 |
| Vtlr 5′ CATCCCCGTACGACTGATCCC | | 16 |
| Vt2f 5′ CGGAATGCAAATCAGTCGTCACTCAC | Verocytotoxin 2 | 17 |
| Vt2r 5′ TCCCCGATACTCCGGAAGCAC | | 18 |
| HlyAf 5′ TGCAGCCTCCAGTGCATCCCTC | The hlyA gene encoding alpha | 19 |
| HlyAr 5′ CTTACCACTCTGACTGCGATCAGC | hemolysin | 20 |
| STaf 5′ GTGAAACAACATGACGGGAGG | Heat-stable enterotoxin 1 | 21 |
| STar 5′ ATAACATCCAGCACAGGCAGG | | 22 |
| STbf 5′ GGGGTTAGAGATGGTACTGCTGGAG | Heat-stable enterotoxin 2 | 23 |
| STbr 5′ GACAATGTCCGTCTTGCGTTAGGAC | | 24 |
| LTf 5′ CCGTGCTGACTCTAGACCCCCA | Heat-labile enterotoxin LT | 25 |
| LTr 5′ CCTGCTAATCTGTAACCATCCTCTGC | | 26 |
| Eaef 5′ CCAGGCTTCGTCACAGTTGCAGGC | The eae gene coding for inti- | 27 |
| Eaer 5′ CGCCAGTATTCGCCACCAATACC | min present in AEECstrains | 28 |
| Enterotoxigenic Bacteroides fragilis (ETBF) | | |
| Bf1F 5′ GTTAGTGCCCAGATGCAGGATGCGG | The genes coding for | 29 |
| Bf2F 5′ GAACTCGGTTTATGCAGTTCATGGACTG | Bacteroides fragilis | 30 |
| Bf3R 5′ TGGGTTGTAGACATCCCACTGGCTT | enterotoxins (BFT1, Bft2 and | 31 |
| Bf4R 5′ GGATACATCAGCTGGGTTGTAGACATCCC | BFT3) | 32 |
| E. coli adhesins | | |
| PapF 5′ CCGGCGTTCAGGCTGTAGCTG | The genes coding for | 33 |
| PapR 5′ GCTACAGTGGCAGTATGAGTAATGACCGTTA | pathogenicity islands (PAI I, | 34 |
| PAI1 5′ TAGCTCAGACGCCAGGATTTTCCCTG | PAI II)¹⁶ and sfp gene | 35 |
| PAI2 5′ CCTGGCGCCTGCGGGCTGACTATCAGGG | cluster | 36 |
| BmaEf 5′ CTAACTTGCCATGCTGTGACAGTA | The bmaE gene for M- | 37 |
| BmaEr 5′ TTATCCCCTGCGTAGTTGTGAATC | agglutinin subunit; Afa-8 | 38 |
| | gene cluster | |
| Sfaf 5′ CGGAGGAGTAATTACAAACCTGGCA | S-fimbrial adhesins encoded | 39 |
| Sfar 5′ CTCCGGAGAACTGGGTGCATCTTAC | by sfaD to sfaE | 40 |
| Afaf 5′ TATGGTGAGTTGGCGGGGATGTACAGTTACA | AfaE-3 gene cluster | 41 |
| Afar 5′ CCGGGAAAGTTGTCGGATCCAGTGT | | 42 |
| AIDA1 5′ TATGCCACCTGGTATGCCGATGAC | The aidA gene coding E. coli | 43 |
| AIDA2 5′ ACGCCCACATTCCCCCAGAC | AIDA-I adhesin in DAEC strains | 44 |
| AggRf 5′ GAGTTAGGTCACTCTAACGCAGAGTTG | The aggR gene for adhesin of | 45 |
| AggRr 5′ GACCAATTCGGACAACTGCAAGCATCTAC | aggregative adherence fimbria | 46 |
| | I | |
| Ag43F 5′TGACACAGGCAATGGACTATGACCG | The agn43 gene coding for | 47 |
| Ag43R 5′GGCATCATCCCGGACCGTGC | antigen involved in E. coli | 48 |
| | autoaggregation | |
| Flagella and type 1 fimbrae typing | | |
| Primer1 5′ CAAGTCATTAATAC(A/C)AACAGCC | The fliC genes coding for | 49 |
| Primer2 5′ GACAT(A/G)TT(A/G)GA | flagellin proteins | |
| (G/A/C)ACTTC(G/C)CT | | 50 |
| | The fimH gene encoding FimH | |
| FimHf 5′ CTGGTCATTCGCCTGTAAAACCGCCA | subunit of type 1 pili | 51 |
| FimHr 5′ GTCACGCCAATAATCGATTGCACATTCCCT | | 52 |
| Ribosomal DNA-based primers | | |
| ITSF 5′ GTCGTAACAAGGTAGCCGTA | 16S-23S rRNA intergenic | 53 |
| ITSReub 5′ GCCAAGGCATCCACC | transcribed spacers | 54 |
| 27f 5′ AGAGTTTGATCMTGGCTCAG | Conserved 16S rDNA used to | 55 |
| 342r 5′ CTGCTGCSYCCCCTAG | amplify ribosomal genes | 56 |

Statistical analysis: For statistical analysis of data we applied a Chi square test based on the Mantel Haenszel method (Epi Info version 6.04, CDC, Atlanta, Ga., USA).

### Results

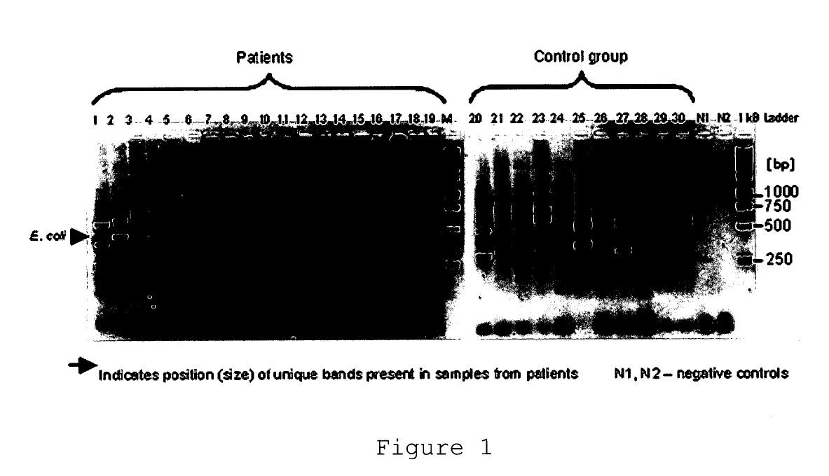

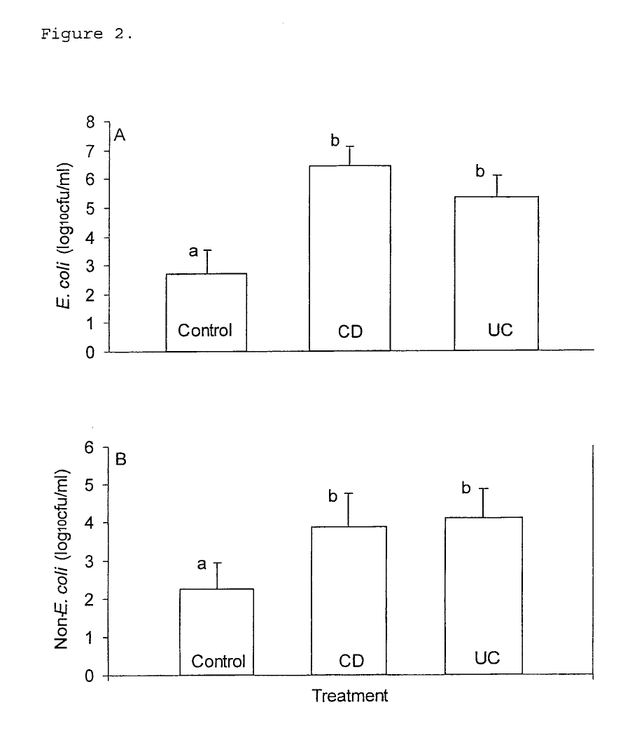

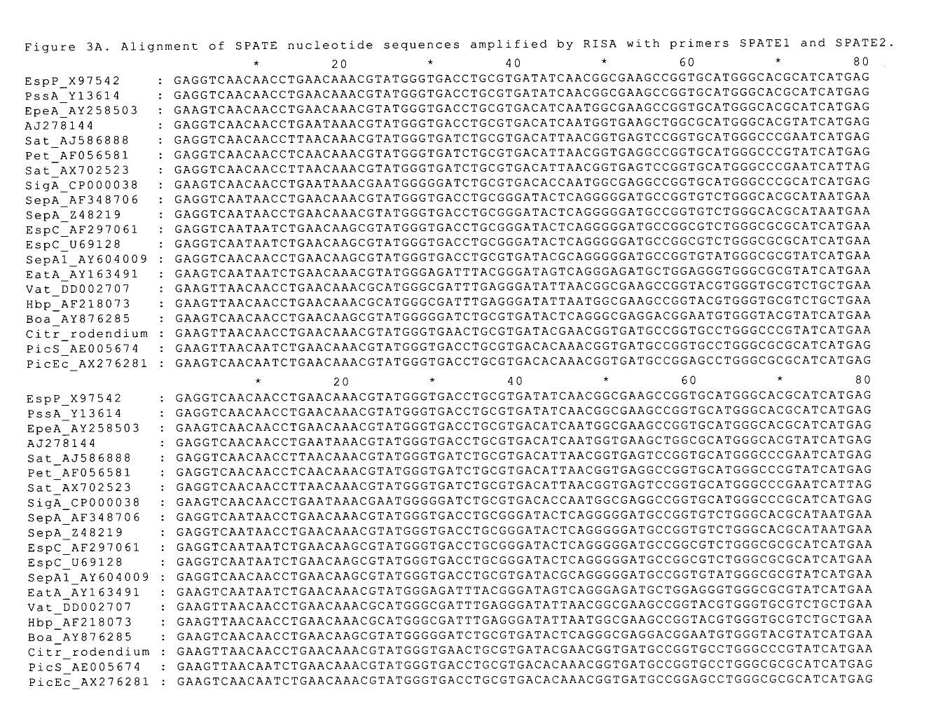

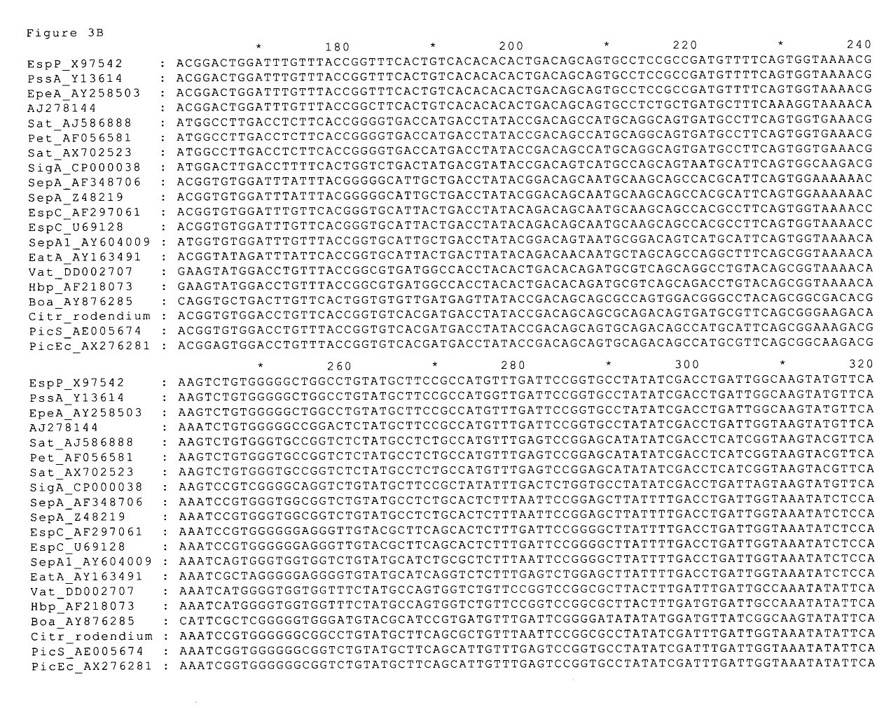

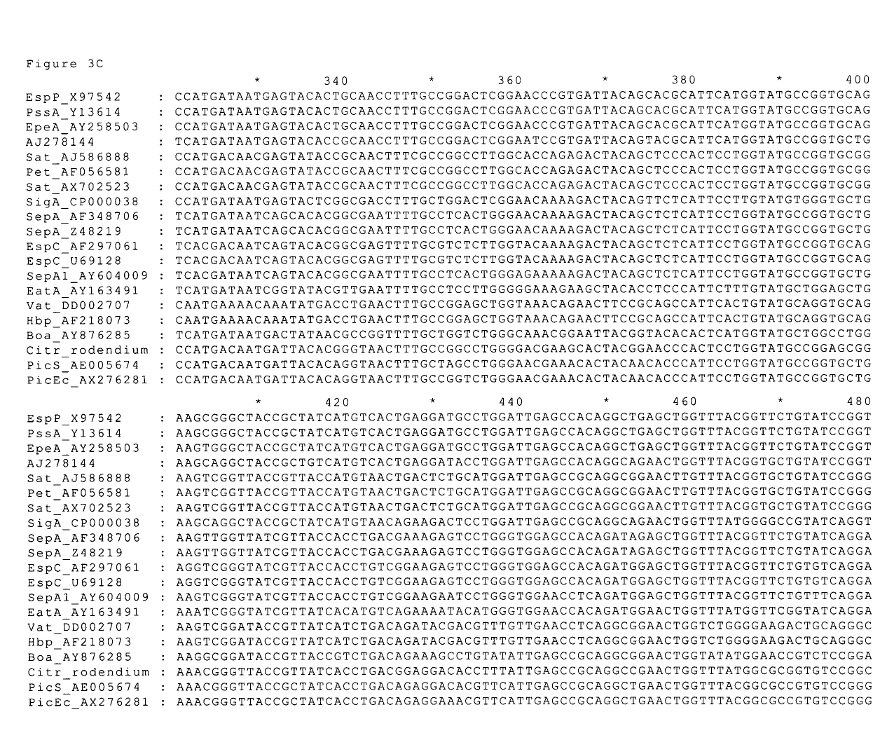

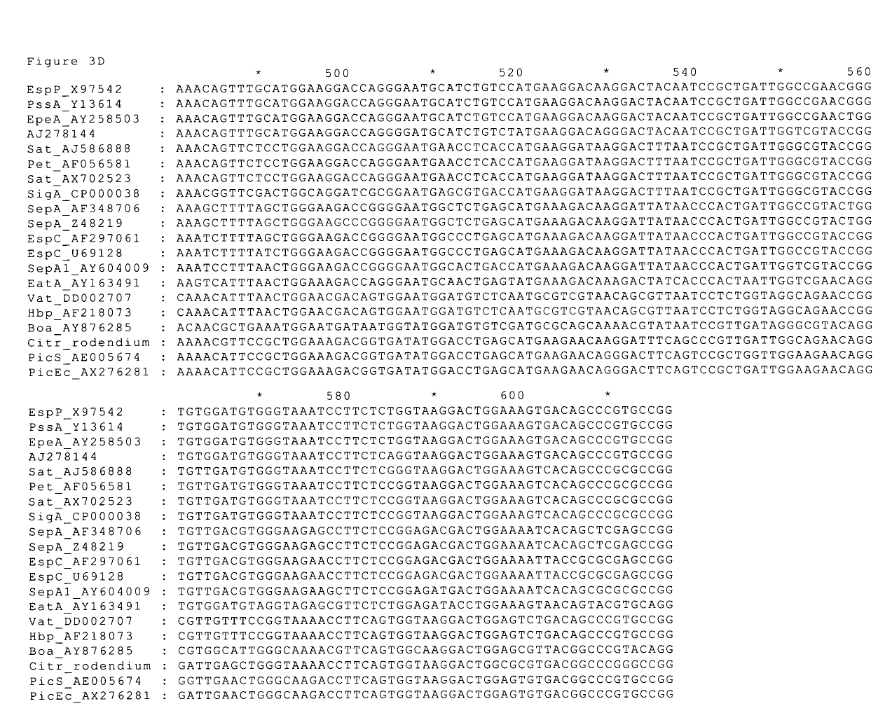

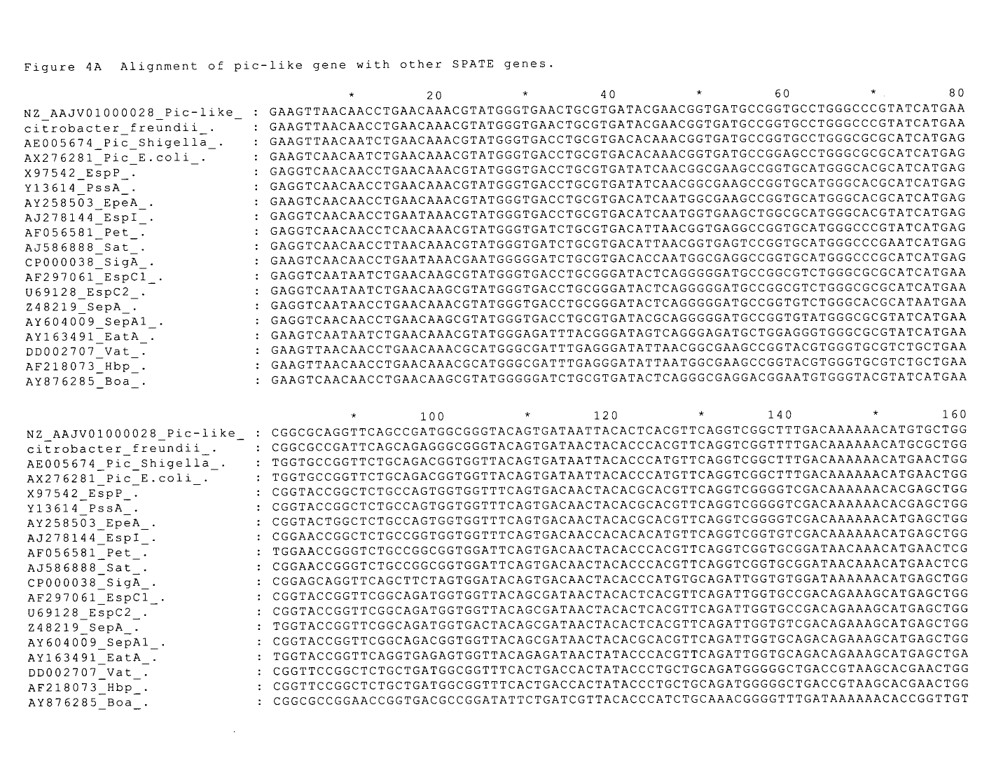

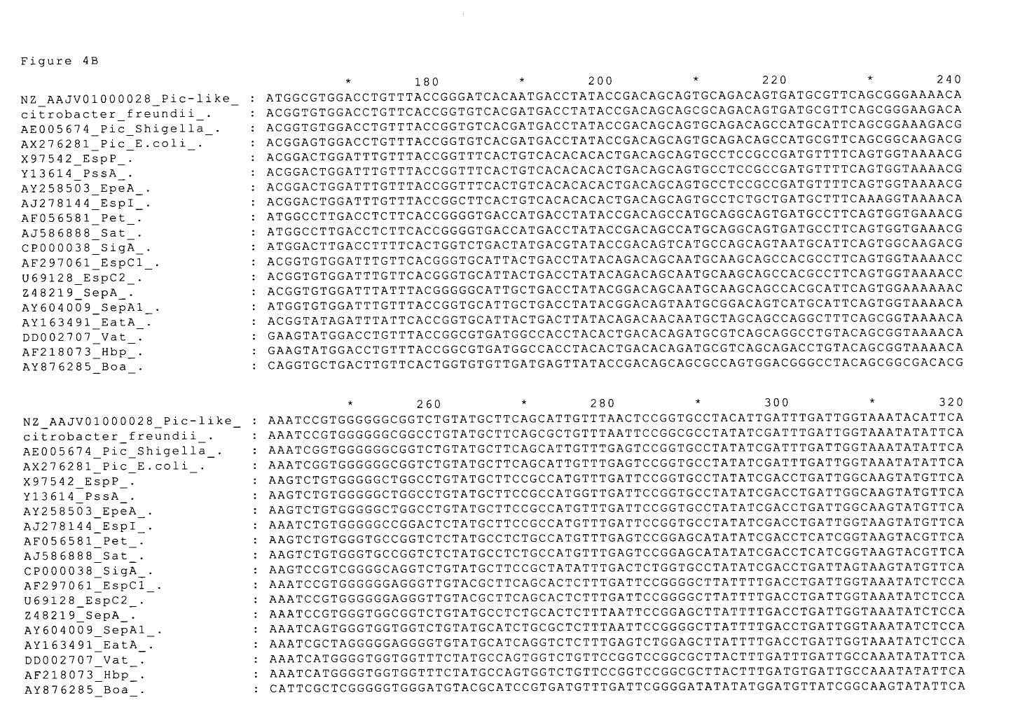

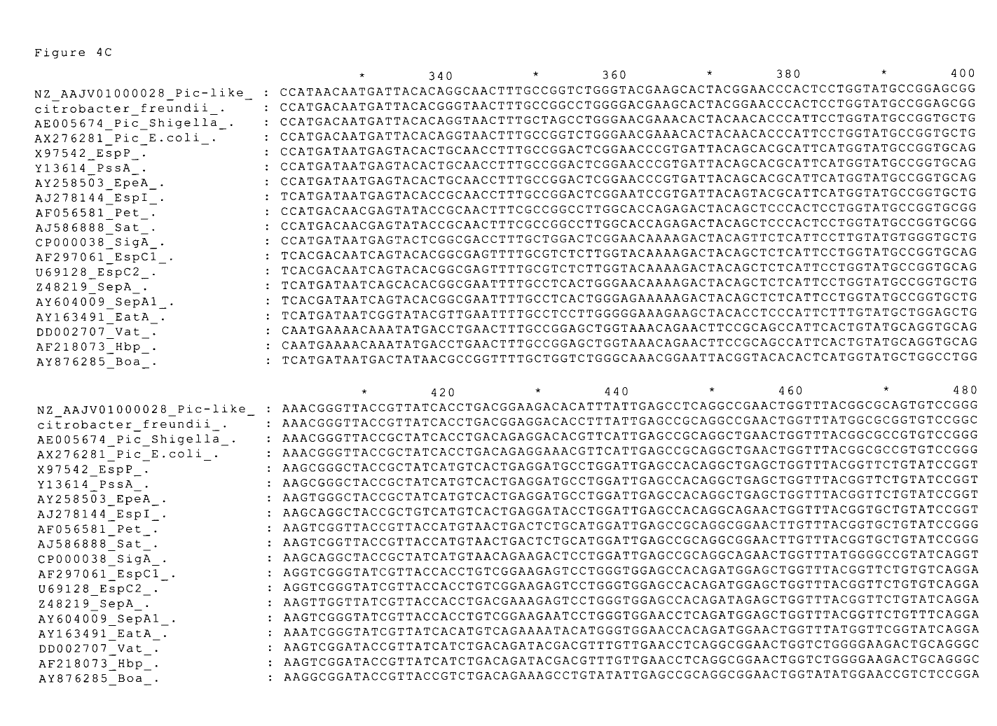

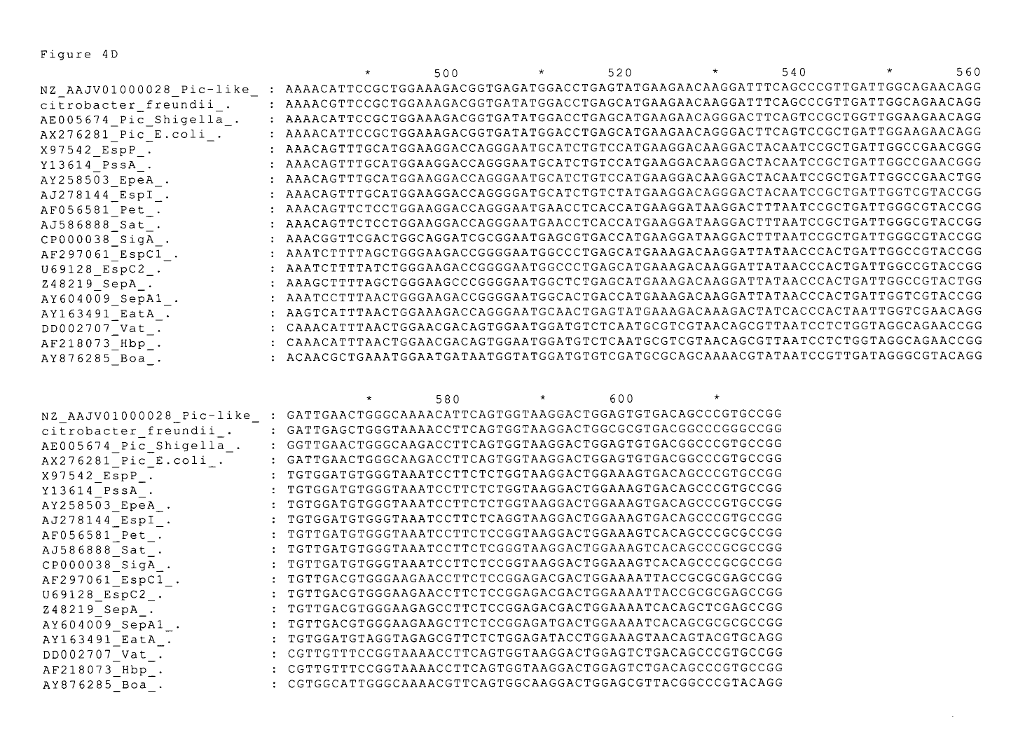

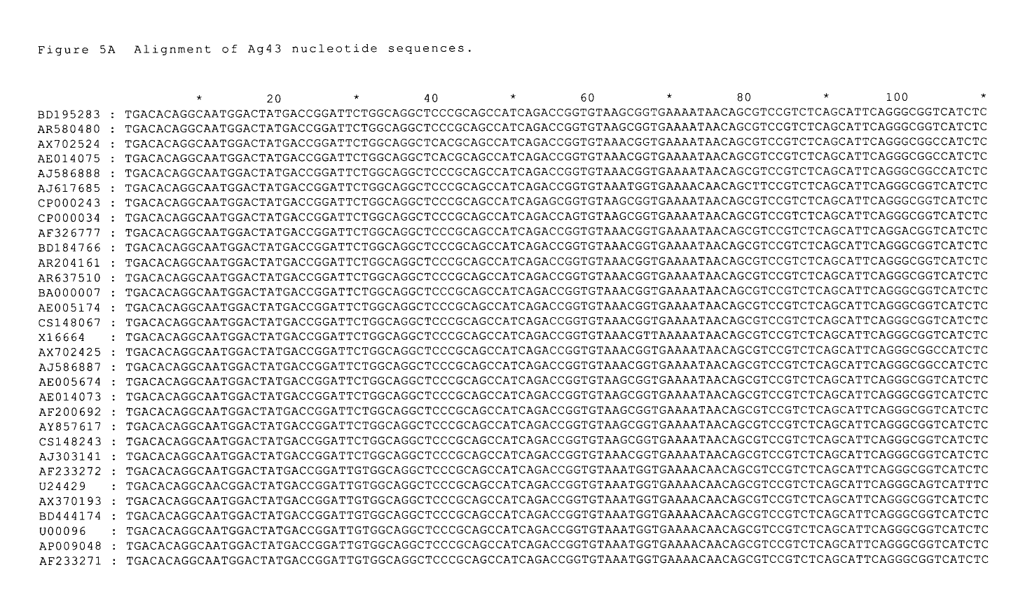

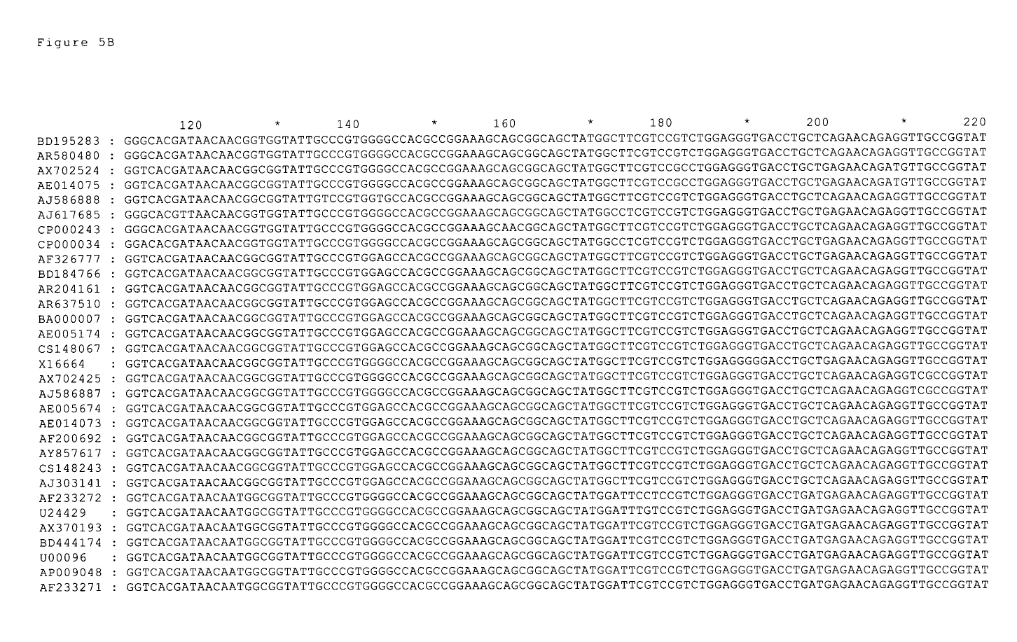

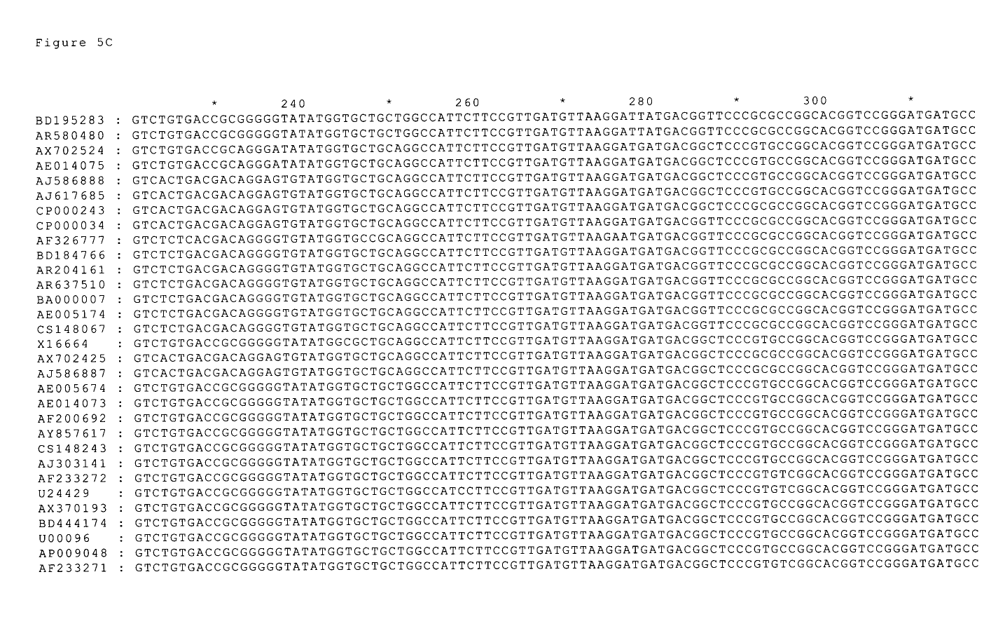

A total of 84 biopsies from 15 controls, 13 Crohn's disease (CD) patients, and 19 ulcerative colitis (UC) patients were undertaken (table 1). In most cases, more than one biopsy was obtained from multiple sites, or adjacent sites from the same subject. DNA was extracted from each biopsy sample and subjected to RISA analysis (FIG. 1). We were able to identify bands (˜450 bp) that were consistently present in approximately 70% of patients but in less than 30% of controls (FIG. 1). The bands in the controls were also of a much lower density than those from IBD tissue. Five bands from IBD tissue were retrieved from the gel, sequenced, and aligned with Genbank sequence and found to be E. coli.

Resuscitation of biopsies by resuspension in buffered peptone water, incubation for 16 h at 37° C., and subsequent plating on chromogenic agar allowed growth of coliform bacteria in over 90% of biopsy tissues. Chromogenic agar enriches for predominantly coliform bacteria but only lactose-fermenting bacteria (E. coli) turn purple. We could culture purple colonies in only 46.7% of control subjects, 69.2% of CD patients and 63.2% of UC patients even though non-E. coli coliforms (white and pink colonies) could be cultured from most biopsies (table 3).

Serial dilution and plating on chromogenic agar allowed for quantification of total coliforms and E. coli (FIG. 2). The numbers of E. coli (4×102/ml) and non-E. coli coliforms (6.3×105/ml) cultured were higher (p<0.05) in CD and UC than in controls. There was a poor correlation (r=0.22) between site of inflammation and presence of E. coli and data was pooled by subject.

TABLE 3
Microbial tissue phenotypes encountered from control and IBD tissues^{a}.

| | | | | | | E. coli |
|---|---|---|---|---|---|---|
| | E. coli^{b} | | | E. coli | E. coli | type 1 |
| Disease Patient | group | Non-E. coli^{c} | SPATE | adhesins | H-type | fimbriae |
| Control 41A | ND | Enterobacter sp. | ND | — | — | — |
| | | Bacteroides | | | | |
| | | fragilis Bft1^{d} | | | | |
| Control 43A | B2 | | Vat | Ag43, I PAI | H7 | bovine |
| Control 48B | B2 | | Vat, Pic-like | Sfp^{e} | H4 | APEC |
| Control 50A | B2 | | Vat, Sat, Pic | Ag43 | H1 | APEC |
| Control 58 | ND | Enterobacter | ND | — | — | — |
| | | flavescens | | | | |
| Control 59B | ND | Klebsiella | ND | — | — | — |
| | | oxytoca | | | | |
| Control 73A | A | Klebsiella sp. | ND | AIDA-I | H12 | bovine |
| Control 69A | ND | Enterococcus sp. | ND | — | — | — |
| | | Bacteroides | | | | |
| | | fragilis Bft1 | | | | |
| Control 76 | ND | Escherichia | SigA, Sat^{g}, SepA | — | — | — |
| | | fergusonii | | | | |
| | | Staphylococcus | | | | |
| | | epidermidis | | | | |
| | | Staphylococcus | | | | |
| | | capitis | | | | |
| | | Bacteroides | | | | |
| | | fragilis Bft2^{d} | | | | |
| Control 80 | ND | Escherichia | SigA, Sat^{g}, SepA | — | — | — |
| | | fergusonii | | | | |
| | | Klebsiella sp. | | | | |
| Control 81 | ND | Enterococcus | ND | — | — | — |
| | | durans | | | | |
| Control 87 | ND | Klebsiella sp. | ND | — | — | — |
| Control 90 | A | | ND | AIDA-I, | H11 | bovine |
| | | | | Ag43 | | |
| Control 17B | D | | ND | Ag43 | H4 | UPEC |
| Control 88 | A | Klebsiella sp. | ND | AIDA-I | H21 | bovine |
| CD 15B | B2 | | Vat, Pic-like | Ag43 | H4 | APEC |
| CD 79A | B2 | | Vat, Sat, Pic | Ag43 | H1 | APEC |
| CD 91A | B1 | Acidovorax sp. | ND | AIDA-I | H21 | bovine |
| CD 118 | ND | Staphylococcus | ND | — | — | — |
| | | auricularis | | | | |
| CD 120A | D | | EspI | PAI I, | H52 | bovine |
| | | | | AIDA-I, | | |
| | | | | Ag43 | | |
| CD 124A | ND | Escherichia | SigA, Sat^{g}, SepA | — | — | — |
| | | fergusonii | | | | |
| | | Enterococcus | | | | |
| | | faecium | | | | |
| | | Bacteroides | | | | |
| | | fragilis Bft1 | | | | |
| CD 125B | ND | Klebsiella | ND | — | — | — |
| | | oxytoca | | | | |
| CD 126C | B2 | | ND | AIDA-I, | H39 | bovine |
| | | | | Ag43 | | |
| CD 132 | B2 | | Sat | AfaE-3, | H4 | bovine |
| | | | | Ag43, | | |
| | | | | AIDA-I | | |
| CD 137 | ND | Klebsiella sp. | ND | — | — | — |
| CD 138 | ND | Staphylococcus | ND | — | — | — |
| | | aureus | | | | |
| | | Pseudomonas | | | | |
| | | putida | | | | |
| | | Klebsiella sp. | | | | |
| CD 146 | B2 | | Vat | Sfa^{f}, | H7 | APEC |
| | | | | Ag43, PAII | | |
| CD 149 | B2 | | Vat, Pic | Sfa, | H5 | APEC |
| | | | | Ag43, PAII | | |
| CD 128 | B2 | Escherichia | Vat, SigA, Sat^{g}, | Sfa | H7 | UPEC |
| | | fergusonii | SepA | | | |
| UC 117A | D | | ND | AIDA-I, | H18 | bovine |
| | | | | Ag43 | | |
| UC 119A | ND | Enterococcus | ND | — | — | — |
| | | faecium | | | | |
| UC 121 | B1 | | ND | ND | H10 | bovine |
| UC 122 | ND | Enterococcus | ND | — | — | — |
| | | faecalis | | | | |
| | | Bacteroides | | | | |
| | | fragilis Bft1 | | | | |
| UC 127A | B2 | Enterobacter | Vat, Sat, Pic | F1C | H7 | bovine |
| | | cloacae | | | | |
| UC 130 | ND | Bacillus | ND | — | — | — |
| | | licheniformis | | | | |
| | | Bacillus pumilus | | | | |
| UC 131A | B2 | Escherichia | Vat, Sat^{g}, Pic, | Ag43, PAII | H10 | APEC |
| | | fergusonii | SigA, Sat, SepA | | | |
| | | Bacteroides | | | | |
| | | fragilis Bft2 | | | | |
| UC 133A | ND | Klebsiella sp. | ND | — | — | — |
| UC 135 | B2 | Klebsiella | Pic | AIDA-I, | H31 | bovine |
| | | oxytoca | | Ag43 | | |
| UC 136 | B2 | ND | Vat | Ag43, PAII | H7 | bovine |
| UC 139 | D | ND | ND | AIDA-I | H6 | APEC |
| UC 140A | ND | Enterococcus sp. | ND | — | — | — |
| UC 141 | B2 | ND | ND | Sfa, | H7 | APEC |
| | | | | Ag43, PAII | | |
| UC 142 | B2 | ND | Vat | AfaE-3, | H5 | APEC |
| | | | | Ag43 | | |
| UC 143 | B2 | ND | Sat, Pic-like | AfaE-3, | H4 | APEC |
| | | | | Ag43 | | |
| UC 145B | B2 | ND | Vat, Pic-like | Ag43 | H4 | bovine |
| UC 147 | B2 | Klebsiella sp. | Vat | Sfa, Ag43 | H5 | APEC |
| UC 134A | ND | Klebsiella sp. | ND | — | — | — |

^{a}A total of 84 biopsies from 47 subjects were used. Data are not shown by site, and were pooled, because the correlation between bacterial species and site, within and between subjects was low (r = 0.22).
^{b}A total of 28 E. coli strains and 34 non-E. coli were picked and assessed. ND = E. coli not detected.
^{c}Identity was determined by sequence the 16S rDNA between with 27f and 342r.
^{d}Bft1, Bft2 are enterotoxins produced by Bacteroides fragilis detected from tissue by nested-PCR method.
^{e}sfp is a gene claster of Sorbitol-Fermenting Enterohemorrhagic E. coli O157:H⁻.
^{f}sfa gene is coding for S-fimbriae minor subunit of E. coli UTI89.
^{g}Sat - gene coding for autotransporteur toxin Sat identified in E. fergusonii.

Five purple colonies were picked from all agar plates (total 135 colonies) that showed E. coli growth. Taxonomic identity of the E. coli was confirmed by reactivity to indole, methyl red, Vogues Proskauer, and citrate utilisation. There were also few polymorphisms in the RISA product, confirming that isolates were predominantly clonal by subject, irrespective of site. We selected 150 colonies of non-E. coli (5 per biopsy) of pink, blue, and white colonies from plates and were able to confirm that none of these colonies were E. coli by checking for reactivity to indole, methyl red, Vogues Proskauer, and citrate utilisation. The identities of these colonies were determined by 16S-rDNA sequence analysis (table 3).

E. coli comprises four phylogenetic groups (A, B1, B2, and D) with virulent types typically belonging to groups B2 and D.15 These groups can be identified by a simple PCR procedure of the chuA, and yjaA genes and a cryptic DNA fragment (table 2). The authors15 indicated that these groups could be identified in a single multiplex PCR for all DNA targets but we did not get reproducible results. We modified the primers (table 2) and the PCR conditions and were able to generate highly reproducible results from a suite of pathogenic and non-pathogenic E. coli in our laboratory (data not shown). The abundance of the pathogenic B2+D groups was significantly (p=0.04) greater in IBD than in controls (table 4).

E. coli were also assayed for the presence of serine protease autotransporter proteins (SPATE). Alignments were made of prominent group (Vat, Sat, Pic, EspI) of SPATE nucleic acid sequences, and primers to conserved regions were designed to amplify targets which, when digested with HaeIII, were diagnostic of the different groups of SPATE. Identity of each SPATE was confirmed by sequence analysis. One new SPATE, Pic-like, was identified by sequence analysis and alignment with known SPATE. When all SPATE-positive E. coli isolates were totalled, IBD patients had a higher number of SPATE-positive isolates than the controls (table 4). Interestingly this SPATE sequence appeared in the enteropathogenic E. coli E22 genome (Genbank: AAJVO1000028) but no functions were assigned.

TABLE 4
Distribution of virulence features of E. coli
isolated from IBD patients and controls

| | Control | UC | CD |
|---|---|---|---|
| | (15 | (19 | (13 |
| | subjects | patients | patients |
| | 28 | 27 | 29 |
| Item¹ | tissues) | tissues) | tissues) |
| Group | | | |
| A | 3 (20)² | 0 | 0 |
| B1 | 0 | 1 (5.3) | 1 (7.7) |
| B2 | 3 (20) | 9 (47.4) | 7 (53.8) |
| D | 1 (6.7) | 2 (10.5) | 1 (7.7) |
| B2 + D | 4 (26.7) | 11 (57.9) | 8 (61.5) |
| SPATE | | | |
| Vat | 3 (20) | 7 (36.8) | 4 (30.8) |
| Pic | 1 (6.7) | 3 (15.8) | 3 (23.1) |
| Sat | 3 (20) | 4 (21.05) | 3 (23.1) |
| Pic-like | 1 (6.7) | 2 (15.4) | 1 (7.7) |
| EspI | 0 | 0 | 1 (7.7) |
| SepA | 2 (13.3) | 2 (10.5) | 1 (7.7) |
| SigA | 2 (13.3) | 2 (10.5) | 1 (7.7) |
| Total | 5 (33.3) | 9 (47.4) | 7 (53.8) |
| Adhesins | | | |
| Ag43³ | 4 (26.7) | 9 (47.4) | 7 (53.8) |
| AfaE3 | 0 | 2 (10.5) | 1 (7.7) |
| AIDA-I | 3 (20) | 3 (15.8) | 4 (30.8) |
| FlC | 0 | 1 (5.3) | 0 |
| Sfa | 0 | 3 (15.8) | 2 (15.4) |
| PAI I | 1 (6.7) | 3 (15.8) | 3 (23.1) |
| Sfp | 1 (6.7) | 0 (5.3) | 0 |
| Total | 7 (46.6) | 12 (63.2) | 8 (61.5) |

¹Functions of each of the genotypic characteristics is given in table 2.
²Values in parentheses represent the frequency of items in patients as percentage.
³Antigen 43 (Ag43) is a self-recognizing surface adhesin found in most Escherichia coli strains.

The PCR analyses of E. coli isolates for a range of adhesins commonly found in pathogenic E. coli were conducted (tables 3, 4). Isolates were only positive for agn43, aida, and gene clusters coding for AfaE-3, FIC, Sfa, PAI I, and Sfp. No E. coli positive for cnf1, cnf2, eae, hlyA, ipgB, aggR, bmaE, or genes coding for verotoxins, heat-stable and heat-labile toxins were found in biopsies of patients with UC or CD.

Chi-squares test based on the Mantel-Haenszel method (table 5) was performed to verify relationships between disease, B2+D genotype, SPATE toxins, and adhesins. We observed a significant difference (p=0.04) in the number of E. coli isolates from the B2+D genotype for both UC and CD patients (Table 5). The relationship between IBD and an adhesin (p=0.07), or two or more adhesins (p=0.03) was significant but not with a SPATE plus an adhesin (p=0.15). The relationship between the B2+D genotype and SPATE9(s), adhesion(s), or APEC was significant (p<0.05).

TABLE 5
Statistical relationship between the presence of
virulence factors in IBD patients and control subjects

| Disease | Virulence factor | p-value |
|---|---|---|
| IBD | 2 or more adhesins | 0.03* |
| IBD | B2 + D | 0.04* |
| IBD | Adhesins | 0.07 |
| IBD | SPATE(s) + adhesin(s) | 0.15 |
| B2 + D genotype | Virulence feature | p-value |
| B2 + D | SPATE(s) + adhesin(s) | 0.002** |
| B2 + D | Adhesins | 0.04* |
| B2 + D | SPATE | 0.02* |
| B2 + D | APEC | 0.04* |

Statistically significant results
*(p < 0.05),
**(p < 0.01)

### Discussion

In our laboratory work, we: (a) use a culture-independent method to identify DNA bands that are present in IBD tissue but not in controls; (b) cut the bands out of the gel and sequence them; (c) identify the bacterial species based on the sequence composition; (d) specifically culture the bacterial group identified from the sequence information, and (e) investigate potential virulence factors

in the cultured bacteria. The RISA analysis (FIG. 1) provided the rationale for culturing E. coli and other Entero-bacteriaceae.

The numbers of Enterobacteriaceae in our biopsy tissues were 3 to 4 logs higher in the IBD tissue than in the controls (FIG. 2). Martin et al (ref 17) only found a significant increase in the numbers of E. coli in CD biopsies, but not UC, after a mucin-releasing step with dithiothreitol. Darfeuille-Michaud et al (ref 20) isolated E. coli in higher numbers from IBD tissue than from controls but did not enumerate the bacteria on the epithelial tissue with techniques to determine numbers in a range greater than one log. Mylonaki et al (ref 21) used fluorescence microscopy to demonstrate that the numbers of E. coli were high in rectal tissue of UC patients but not of controls, but this methodology did not allow for decimal enumeration.

Conte et al (ref 22) could demonstrate that gram-negative bacteria, including E. coli increased by 3 to 4 logs in IBD tissue, a result strikingly similar to ours. It is thus clear that the numbers of Enterobacteriaceae do increase on the epithelial tissue of IBD patients, irrespective of the differences in techniques used. The major difference in our study was that we resuscitated tissue in buffered peptone water to ensure that bacteria, even at very low numbers, were given the maximum chance to grow. This was done given the fact that E. coli in environmental samples enter into the viable, but non-culturable state, making them difficult to grow without a resuscitation step (ref 23).

Clermont et al (ref 15) developed a method to type pathogenic E. coli using chuA, a gene required for heme transport in enterohaemorrhagic 0157:H7 E. coli, yjaA, a gene identified in E. coli K-12 but which has no known function, and TSPE4.C2, a cryptic fragment that was identified from subtractive libraries. These genes, when applied to 230 isolates, determined that types B2, and to a lesser extent D, included virulent extraintestinal strains of E. coli but the prevalence of B2 and D in gastro-intestinal isolates was not determined. E. coli from stool samples from a range of geographically separate normal healthy human subjects determined that non-pathogenic groups A and B1 were most prevalent, while group D only made up 15%, and group B2 11% of isolates.24 A significant relationship (p <0.05) between the B2 genotype of “resident”, or adherence factor carrying E. coli in infants25 and in human colonic cells of adults26 has been demonstrated. We demonstrated a significant relationship (p=0.04) between IBD and the B2+D genotype (table 5). Our data suggest a significant relationship between the presence of a SPATE (p=0.02), or an adhesin (p=0.04), in a B2+D positive E. coli strains.

The “resident” population that Nowrouzian et al (ref 25) referred to are strains that have adherence factors, in particular P fimbriae that promote adherence to enterocytes. This definition is of course somewhat arbitrary because there are a large number of cell factors that promote adhesin to intestinal tissue. E. coli from our controls had relatively few adhesins (table 4), while IBD strains had a higher prevalence of Ag43, AIDA-I, Sfa, AfaE-3, and PAI I. Ag43 is a surface adhesin that promotes bacterial biofilm formation due to cell-to-cell aggregation (ref 27), Sfa is one of a class of S-fimbrial adhesins (ref 16), and AIDA-I is an adhesin-like protein (ref 16). Martin et al (ref 17) measured adherence and invasion of IBD-derived E. coli in tissue culture, and showed that E. coli strains isolated from Crohn's disease patients possessed haemagglutinating ability to all red cells regardless of blood group. We did not measure adherence and invasion in a cell culture assay but all E. coli isolates both from IBD tissues and control group were negative for the presence of bmaE gene encoding M-agglutinin.

A novel finding of this study is the higher prevalence of Escherichia coli from the B2+D phylogenetic group in IED tissues. Moreover, SPATE (ref 28) plus adhesins which have been primarily associated with E. coli isolated from urinary tract infections, were also more prevalent in E. coli isolates from IBD patients. SPATE is a unique class of transporter found in the Enterobacteriaceae that direct their own transport across the outer cell membrane. These proteins have been implicated in virulence but their precise role is not known. We believe that the functional properties of SPATE make them potentially important in IBD because they exhibit functions like degradation of the barrier function of the gut, and cleavage of proteins in the enterocyte, all of which are phenotypes associated with IBD (ref 28). For example Vat, Pic, and Pic-like have haemaglutinin, mucinase, and elastase activity (ref 29, 30), Sat has cytotoxic effects (ref 31-33) on cells as well as elastase activity (ref 27-30), and EspP cleaves lipoproteins (ref 27-30). More recently, functional studies on SPATE have demonstrated that Pic and Sat can cleave coagulation factor V, potentially linking it to haemorrhagic events in the gut. Additionally, Pic is thought be involved in colonisation of E. coli to intestinal tissue (ref 34).

One means by which SPATE may be involved in promoting inflammation could be as an accessory protein in pathogenicity islands. EspC, a SPATE from enteroaggregative E. coli (EAEC) (ref 35) and the non-SPATE Tsh autotransporter (ref 36) have been associated with PAI. Bidet et al (ref 37) suggested that PAI IJ96 were associated with hra, hlyA, cnf1, and pap. We determined the prevalence of hlyA and cnf1 and cnf2 in our isolates but they were not present. When we designed primers that were conserved for a range of PAI, only 23.1% of CD and 15.8% of UC were positive. If these were the only PAI, then less than 50% of our SPATE would be associated with PAI. Future work would investigate the possibility of the association of other PAI with SPATE.

One might argue that IBD is caused by a dysfunctional immune system that results in an increase in E. coli on the gut tissue, and that E. coli has nothing whatever to do with initiating inflammation in UC or CD. If this was true then the genotypes of E. coli isolated from tissue should be a consequence of random colonisation of gut mucosa. Consequently the distribution of these genotypes should be equivalent among IBD and control tissues. This is not the case, with IBD having more (p=0.04) putatively pathogenic bacteria than controls. What we do not know at this point is whether the increase in certain types of E. coli in IBD is a consequence of inflammation, or the cause. One way forward is to describe the E. coli population before inflammation sets in, for example, obtain biopsy tissue that ranges along the colon, distal and proximal to UC lesions.

The present invention is not to be limited in scope by the specific embodiments described herein. Various modifications of the invention in addition to those described herein will become apparent to those skilled in the art from the foregoing description and accompanying figures. Such modifications are intended to fall within the scope of the claims.

The present invention is directed to each individual feature, system, material and/or method described herein. In addition, any combination of two or more such features, systems, materials and/or methods, provided that such features, systems, materials and/or methods are not mutually inconsistent, is included within the scope of the present invention.

In the specification and claims, all transitional phrases or phrases of inclusion, such as “comprising,” “including,” “carrying,” “having,” “containing,” “composed of,” “made of,” “formed of,” “involving” and the like shall be interpreted to be open-ended, i.e. to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.” The expression “A or B”, unless clearly indicated to the contrary, should be understood to mean “A or B or both”.

Various publications are cited herein, the disclosures of which are incorporated by reference in their entirety or in pertinent part, as is understood from the context of the publication being cited. In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

### REFERENCES

1. Koutroubakis I E. Therapy insight: vascular complications in patients with inflammatory bowel disease. Nat Clin Pract Cardiovasc Med 2005;2:266-272.
2. Sartor R B. Role of commensal enteric bacteria in the pathogenesis of immune-mediated intestinal inflammation: lessons from animal models and implications for translational research. J Pediatr Gastroenterol Nutr 2005;40 (Suppl 1):S30-S31.
3. Rath H C. The role of endogenous bacterial flora: bystander or the necessary prerequisite? Eur J Gastroenterol Hepatol 2003;15:615-620.
4. Bernstein C N, Wajda A, Blanchard J F. The clustering of other chronic inflammatory diseases in inflammatory bowel disease: a population-based study. Gastroenterol 2005;129:827-836.
5. Garcia-Rodriguez L A, Gonzalez-Perez A, Johansson S, et al. Risk factors for inflammatory bowel disease in the general population. Aliment Pharmacol Ther 2005;22:309-315.
6. Hume G, Radford-Smith, G L. The pathogenesis of Crohn's disease in the 21st century. Pathology 2002;34:561-567.
7. Silverberg M S, Satsangi J, Ahmad T, et al. Toward an integrated clinical, molecular and serological classification of inflammatory bowel disease: Report of a Working Party of the 2005 Montreal World Congress of Gastroenterology. Can J Gastroenterol 2005; Suppl A:5-36.
8. Korzenik J R. Past and current theories of etiology of IBD: toothpaste, worms, and refrigerators. J Clin Gastroenterol Nutr 2005;40 (Suppl 1):S30-S31.
9. Bull T J, McMinn E J, Sidi-Boumedine K, Skull A, et al. Detection and verification of Mycobacterium avium subsp. paratuberculosis in fresh ileocolonic mucosal biopsy specimens from individuals with and without Crohn's disease. J Clin Microbiol 2003;41:2915-2923.
10. Eckburg P B, Bik E M, Bernstein C N, et al. Diversity of the human intestinal microbial flora. Science 2005; 308:1635-1638.
11. Riesenfeld C S, Schloss P D, Handelsman J, et al. Metagenomics: genomic analysis of microbial communities. Annu Rev Genet 2004;38:525-552.
12. Rappe M S, Giovannoni S J. The uncultured microbial majority. Annu Rev Microbiol 2004;57:369-394.
13. Cardinale M, Brusetti L, Quatrini P, et al. Comparison of different primer sets for use in automated ribosomal intergenic spacer analysis of complex bacterial communities. Appl Environ Microbiol. 2004;70:6147-56.
14. Bernstein C N, Nayar G, Hamel A, et al. Study of animal-borne infections in the mucosas of patients with inflammatory bowel disease and population-based controls. J Clin Microbiol 2003;41:4986-4990.
15. Clermont O, Bonacorsi S, Bingen E, et al. Rapid and simple determination of the Escherichia coli phylogenetic group. Appl Environ Microbiol 2000;66:4555-4558.
16. Schmidt H, Hensel M. Pathogenicity islands in bacterial pathogenesis. Clin Microbiol Rev 2004;17:14-56.
17. Martin H M, Campbell B J, Hart C A, et al. Enhanced Escherichia coli adherence and invasion in Crohn's disease and colon cancer. Gastroenterology. 2004;127:80-93.
18. Machado J, Grimont F, Grimont P A, et al. Identification of Escherichia coli flagellar types by restriction of the amplified fliC gene. Res Microbiol. 2000;151:535-546.
19. Lane DJ. 16S/23S rRNA sequencing. Nucleic acid techniques in bacterial systematics. E. Stackebrandt and M. Goodfellow, eds. New York, N.Y., John Wiley and Sons: 115-175.
20. Darfeuille-Michaud A, Boudeau J, Bulois P, et al. High prevalence of adherent-invasive Escherichia coli associated with ileal mucosa in Crohn's disease. Gastroenterology 2004;127:412-421.
21. Mylonaki M, Rayment N B, Rampton D S, et al. Molecular characterization of rectal mucosa-associated bacterial flora in inflammatory bowel disease. Inflamm Bowel Dis 2005;11:481-487.
22. Conte M P, Schippa S, Zamboni I, et al. Gut-associated bacterial microbiota in paediatric patients with inflammatory bowel disease. Gut 2006 Apr 28; [Epub ahead of print].
23. Nystrom T. Not quite dead enough: on bacterial life, culturability, senescence, and death. Arch Microbiol 2001;176:159-164.
24. Duriez P, Clermont O, Bonacorsi S, et al. Commensal Escherichia coli isolates are phylogenetically distributed among geographically distinct human populations. Microbiology 2001;147:1671-1676.
25. Nowrouzian F L, Wold A E, Adlerberth I. Escherichia coli strains belonging to phylogenetic group B2 have superior capacity to persist in the intestinal microflora of infants. J Infect Dis 2005;191:1078-1083.
26. Nowrouzian F L, Adlerberth I, Wold A E. Enhanced persistence in the colonic microbiota of Escherichia coli strains belonging to phylogenetic group B2: role of virulence factors and adherence to colonic cells. Microbes Infect 2006;8:834-840.
27. Henderson I R, Navarro-Garcia F, Desvaux M, et al. Type V Protein Secretion Pathway: the Autotransporter Story. Microbiol Mol Biol Rev 2004;68:692-744.
28. Henderson I R, Nataro J P. Virulence functions of autotransporter proteins. Infect Immun 2001;69:1231-1243.
29. Parham N J, Pollard S J, Desvaux M, et al. Distribution of the serine protease autotransporters of the Enterobacteriaceae among extraintestinal clinical Isolates of Escherichia coli. J Clin Microbiol 2005;43:4076-4082.
30. Dutta P R, Cappello R, Navarro-Garcia F, et al. Functional comparison of serine protease autotransporters of enterobacteriaceae. Infect Immun 2002;70:7105-7113.
31. Betis F, Brest P, Hofman V, et al. The Afa/Dr adhesins of diffusely adhering Escherichia coli stimulate interleukin-8 secretion, activate mitogen-activated protein kinases, and promote polymorphonuclear transepithelial migration in T84 polarized epithelial cells.Infect Immun. 2003;71:1068-74.
32. Taddei C R, Fasano A, Ferreira A J, et al. Secreted autotransporter toxin produced by a diffusely adhering Escherichia coli strain causes intestinal damage in animal model assays. FEMS Microbiol Lett. 2005; 250:263-9.
33. Guignot J, Chaplais C, Coconnier-Polter M H, et al. The secreted autotransporter toxin, Sat, functions as a virulence factor in Afa/Dr diffusely adhering Escherichia coli by promoting lesions in tight junction of polarized epithelial cells. Cell Microbiol. 2006; [Epub ahead of print]
34. Henderson I R, Czeczulin J, Eslava C, et al. Characterization of pic, a secreted protease of Shigella flexneri and enteroaggregative Escherichia coli. Infect Immun 1999;67:5587-5596.
35. Mellies J L, Navarro-Garcia F, Okeke I, et al. EspC pathogenicity island of enteropathogenic Escherichia coli encodes an enterotoxin. Infect Immun 2001;69: 315-324.
36. Dozois C M, Dho-Moulin M, Bree A, et al. Relationship between the Tsh autotransporter and pathogenicity of avian Escherichia coli and localization and analysis of the Tsh genetic region. Infect Immun 2000;68:4145-4154.
37. Bidet P, Bonacorsi S, Clermont O, et al. Multiple insertional events, restricted by the genetic background, have led to acquisition of pathogenicity island IIJ96-like domains among Escherichia coli strains of different clinical origins. Infect Immun 2005;73:4081-4087.
38. Maroncle et al. Infect Imm 2006; 74(11): 6124-6134.
39. Sherlock et al. J Bacteriology 2006; 188(5):1798-1807.

1. A method for diagnosing inflammatory bowel disease (IBD) or determining susceptibility to developing IBD in a subject, the method comprising the step of assaying for serine protease autotransporter (SPATE) or antigen 43 (Ag43) or both, in an enteric bacteria-containing sample from the subject, wherein the presence of SPATE in the sample indicates that the subject has IBD or is susceptible to developing IBD.

2. The method according to claim 1 wherein the subject has symptoms of IBD, or is suspected of having IBD.

3. The method according to claim 1 wherein the IBD is ulcerative colitis (UC) or Crohn's disease (CD).

4. The method according to claim 1, wherein SPATE is assayed by detecting SPATE nucleic acid and wherein Ag43 is assayed by detecting Ag43 nucleic acid.

5. (canceled)

6. The method according to claim 4 wherein the assay for SPATE nucleic acid or for Ag43 nucleic acid comprises detecting a region of SPATE or a region of Ag43 which is conserved among enteric E. coli.

7. The method according to claim 4 wherein the assay for SPATE nucleic acid or for Ag43 nucleic acid comprises detecting a region of SPATE or a region of Ag43 which is specific to enteric E. coli.

8. The method according to claim 4 wherein the assay for SPATE nucleic acid or for Ag43 nucleic acid comprises detecting a region of SPATE or a region of Ag43 which is conserved among virulent enteric strains of E. coli.

9. The method according to claim 4 wherein the assay for SPATE nucleic acid or for Ag43 nucleic acid comprises detecting a region of SPATE or a region of Ag43 which is specific to virulent enteric strains of E. coli.

10. The method according to claim 4 wherein the assay for SPATE nucleic acid or for Ag43 nucleic acid comprises detecting a region of SPATE or a region of Ag43 which is specific to E. coli of the B2 or D genotype or the B2+D genotype.

11. The method according to claim 4 comprising detecting SPATE nucleic acid or detecting Ag43 nucleic acid, or both, by polymerase chain reaction (PCR).

12. The method according to claim 1 comprising assaying for enteric bacterial SPATE or enteric bacterial Ag43 polypeptide or both.

13. The method according to claim 12 wherein the SPATE or Ag43 or both are of enteric E. coli.

14. The method according to claim 13 wherein the SPATE or Ag43 or both are of virulent enteric strains of E. coli.

15. The method according to claim 13 wherein the SPATE or Ag43 or both are of E. coli of the B2 or D genotype or the B2+D genotype.

16. The method according to claim 15 comprising an immunoassay for the SPATE or the Ag43 polypeptide or both.

17. The method according to claim 1 comprising the steps of:
(i) contacting an enteric bacteria-containing sample from a subject with an antibody immunospecific against the SPATE or against the Ag43, or with antibodies against both SPATE and Ag43, under conditions suitable to form a complex between the SPATE or the Ag43 and the antibody; and
(ii) detecting presence or absence of the complex;
wherein presence of the complex indicates that the subject has or is susceptible to developing IBD.

18. The method according to claim 1 wherein the level of SPATE or Ag43 or both in the sample is determined, whereby a higher level of SPATE or Ag43 or both compared to a cut-off value indicates that the subject has IBD or or is susceptible to developing IBD.

19. The method according to claim 1 wherein the subject is human.

20. The method according to claim 1 wherein the sample is a gut tissue biopsy, a sample of intestinal mucosa, stool sample, or intestinal wash.

21. The method according to claim 20 wherein the sample is a colonoscopy tissue biopsy from the lower gastrointestinal (GI) tract.

22-71. (canceled)
